(19) United States
(12) Patent Application Publication
(10) Pub. No.: US 2025/0107897 A1
(43) Pub. Date: Apr. 3, 2025

(54) PATIENT-SPECIFIC SURGICAL POSITIONING GUIDES AND METHODS OF MAKING AND USING THE SAME

(71) Applicant: Carlsmed, Inc., Carlsbad, CA (US)

(72) Inventor: Jeremy Winston, San Diego, CA (US)

(21) Appl. No.: 18/905,055

(22) Filed: Oct. 2, 2024

Related U.S. Application Data
(60) Provisional application No. 63/542,264, filed on Oct. 3, 2023.

(51) Int. Cl. A61F 2/30; A61B 1/317; A61F 2/44; A61F 2/46

(52) U.S. Cl. CPC A61F 2/30942; A61F 2/44; A61F 2/4611; A61B 1/317; A61F 2002/3008; A61F 2002/4633; A61F 2002/4687

(57) ABSTRACT

The present technology includes medical treatment systems having patient-specific interbody implants and patient-specific positioning guides. The patient-specific interbody implant can be specifically designed to permanently occupy a first target location in the patient, and the patient-specific positioning guide can be specifically designed to temporarily occupy a second target location in the patient. Both the patient-specific interbody implant and the patient-specific positioning guide can be coupled to the same inserter instrument at a predetermined spatial orientation such that when the patient-specific positioning guide is positioned at the second target location, the patient-specific interbody implant is automatically directed into the first target location. Methods for designing and manufacturing patient-specific implants and positioning guides are also described herein.

### CROSS-REFERENCE TO RELATED APPLICATION

The present application claims priority to U.S. Provisional Patent Application No. 63/542,264, filed Oct. 3, 2023, the disclosure of which is incorporated by reference herein in its entirety.

### TECHNICAL FIELD

The present disclosure is generally related to medical care, and more particularly to patient-specific surgical positioning guides, including systems and methods designing and manufacturing the same.

### BACKGROUND

Surgical procedures to implant orthopedic implants are used to correct numerous different maladies in a variety of contexts, including spine surgery, hand surgery, shoulder and elbow surgery, total joint reconstruction (arthroplasty), skull reconstruction, pediatric orthopedics, foot and ankle surgery, musculoskeletal oncology, surgical sports medicine, and orthopedic trauma. Spine surgery itself may encompass a variety of procedures and targets, such as one or more of the cervical spine, thoracic spine, lumbar spine, or sacrum, and may be performed to treat a deformity or degeneration of the spine and/or related back pain, leg pain, or other body pain. Common spinal deformities that may be treated using an orthopedic implant include irregular spinal curvature such as scoliosis, lordosis, or kyphosis (hyper- or hypo-), and irregular spinal displacement (e.g., spondylolisthesis). Other spinal disorders that can be treated using an orthopedic implant include osteoarthritis, lumbar degenerative disc disease or cervical degenerative disc disease, lumbar spinal stenosis, and cervical spinal stenosis.

### BRIEF DESCRIPTION OF THE DRAWINGS

The accompanying drawings illustrate various embodiments of systems, methods, and embodiments of various other aspects of the disclosure. Any person with ordinary skill in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1A is a perspective view of medical treatment system configured in accordance with embodiments of the present technology.

FIG. 1B is a first perspective view of a positioning guide of the system of FIG. 1A, with other features of the system omitted for clarity.

FIG. 1C is a second perspective view of the positioning guide of the system of FIG. 1A, rotated 180 degrees relative to the first perspective view of FIG. 1B.

FIG. 2A is a perspective view of a stage of a surgical procedure in which the medical treatment system of FIG. 1A is used to deliver the implant into a patient's spine in accordance with embodiments of the present technology.

FIG. 2B is a top view of the stage of the surgical procedure shown in FIG. 2A.

FIG. 2C is a side view of the stage of the surgical procedure shown in FIG. 2A.

FIG. 3 is a network connection diagram illustrating a system for providing patient-specific medical care in accordance with embodiments of the present technology.

FIG. 4 illustrates a computing device suitable for use in connection with the system of FIG. 1A in accordance with embodiments of the present technology.

FIG. 5 is a flow diagram illustrating a method for providing patient-specific medical care in accordance with embodiments of the present technology.

### DETAILED DESCRIPTION

The present technology includes patient-specific positioning guides and methods of using patient-specific positioning guides to implant a medical device in a patient. The positioning guides can be attached to an inserter instrument, and the inserter instrument can be releasably attached to a medical device, such as an implant, such that a user can manipulate a position of the implant within a patient's body using the inserter instrument. The inserter instrument can therefore deliver and/or deploy the implant within a patient. For example, in spinal procedures, spinal implants (e.g., straight implants, crescent shaped implants, etc.) can be inserted into a disc space. The positioning guide can be sized and/or shaped to mate with a specific target location (e.g., an anterior, posterior, or lateral margin of a vertebral endplate). Of note, the positioning guide and the implant can have a predetermined three-dimensional spatial orientation relative to one another, such that positioning the positioning guide at its target location automatically directs the implant to occupy its target location. Once the implant is inserted and the positioning guide is mated with the target implant location, the inserter instrument and positioning guide can be removed. A user can reengage the inserter instrument with the implant for repositioning and/or removal of the implant, if necessary.

As described in greater detail throughout this Detailed Description, the positioning guide can include one or more contoured surfaces and/or a depth stop such that the positioning guide ensures the implant is inserted at an intended implant location and at an intended implant angle or orientation. Accordingly, in some embodiments the size and shape of the positioning guide is patient specific. For example, the one or more contoured surfaces can be specifically designed to match the contours of patient anatomy (e.g., to mate with patient anatomy at margins of the vertebral bodies). Examples of patient-specific positioning guides are described in detail throughout this Detailed Description, including in Section A below.

In some embodiments, the present technology includes methods of designing and manufacturing the patient-specific positioning guides. The method can include designing patient-specific positioning guides for use with a particular patient-specific implant specifically designed to occupy a specific location within a particular patient. Examples of methods of designing and manufacturing patient-specific positioning guides are described in detail throughout this Detailed Description, including in Section B below.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

As used herein and in the appended claims, the singular forms “a,” “an,” and “the” include plural references unless the context clearly dictates otherwise.

Although the disclosure herein primarily describes systems and methods for treatment planning in the context of orthopedic surgery, the technology may be applied equally to medical treatment and devices in other fields (e.g., other types of surgical practice). Additionally, although many embodiments herein describe systems and methods with respect to implanted devices, the technology may be applied equally to other types of medical devices (e.g., non-implanted devices).

The headings are provided for convenience only and should not be used to interpret the scope of the present technology.

### A. PATIENT-SPECIFIC TREATMENT SYSTEMS, INCLUDING PATIENT-SPECIFIC POSITIONING GUIDES

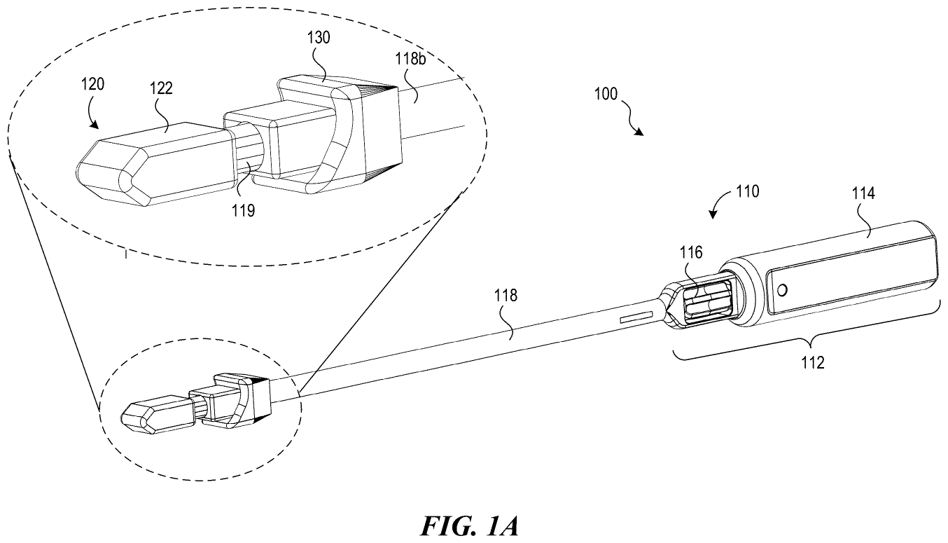

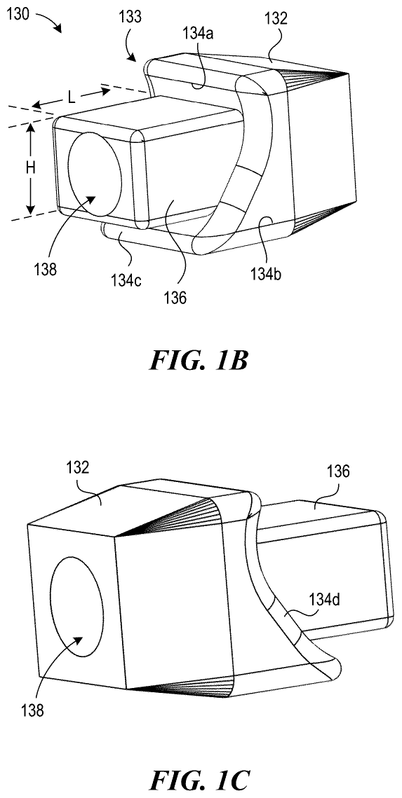

FIG. 1A is a perspective view of a patient treatment system 100 (“the system 100”) configured in accordance with embodiments of the present technology. The system 100 includes an inserter instrument 110, an implant 120, and surgical positioning guide 130. In operation, and as described in greater detail below, the inserter instrument 110 can be used to deliver the implant 120 to, and/or deploy the implant 120 at, a target implant location within a patient.

The inserter instrument 110 can include a proximal complex 112 having a handle 114 and a dial 116. The handle 114 can be sized and shaped to permit a surgeon or other user to control the inserter instrument 110 by grasping the handle 114. The dial 116 can be rotatable relative to the handle 114. As described in greater detail below, a user can rotate the dial 116 to selectively disengage the implant 120 from the inserter instrument 110 (e.g., once the implant 120 is positioned at a desired implant location within the patient).

The inserter instrument 110 can further include an elongated shaft 118 extending distally form the proximal complex 112. The elongated shaft 118 can be composed of a generally rigid material or blend of materials, including, but not limited to, Nitinol, steel, stainless steel, titanium, or other suitable metals or metal alloys. The inserter instrument 110 can further include an inner shaft or rod 119 that extends coaxially within the elongated shaft 118. In some embodiments, the inner rod 119 extends at least between a distal end 118b of the elongated shaft 118 and the proximal complex 112, although in other embodiments the inner rod 119 extends only partially through the elongated shaft 118. Regardless, the inner rod 119 can have a threaded connection (e.g., a threaded male element, not shown) that is sized and shaped to be releasably and threadably coupled to the implant 120 (e.g., via a corresponding threaded female element on the implant 120, not shown). The dial 116 can be rotatably keyed to the inner rod 119. Thus, as described in greater detail below, a surgeon can disengage the implant 120 from the inserter instrument 110 after the implant 120 has been positioned at the target implant location by rotating the dial 116, which can cause the inner rod 119 to rotate. Because the implant 120 will be prevented from rotating due to its placement within the patient, the inner rod 119 will rotate relative to the implant 120, causing the threaded connection therebetween to disengage.

The implant 120 can be any medical device configured for placement within a patient. For example, in some embodiments the implant 120 is an interbody implant configured to be placed within the intervertebral space between adjacent vertebral bodies within a patient's spine. Interbody implants can include, but are not limited to, cages or other fusion devices, artificial discs, spacers, expandable cages or fusion devices, fixation devices, or the like. In other embodiments, the implant 120 can be another spinal implant, such as a plate, a rod, a bracket, a scaffold, an anchor, a connector, or the like.

In some embodiments, the implant 120 is a patient-specific implant designed to be implanted (e.g., permanently implanted) at a specific anatomical location within the patient (referred to herein as “a first target location”). As used herein, the term “patient-specific” refers to an implant 120 that has one or more properties specifically designed for a particular patient. For example, the implant 120 can be designed to have a topography that matches or mates with a corresponding topography of portions of the vertebral bodies between which the implant 120 will be deployed. For example, the implant 120 can have a first (e.g., superior) surface 122 having a first patient-specific topography or shape, and a second (e.g., inferior) surface 124 having a second patient-specific topography or shape. The first patient-specific topography or shape can match a topography of a portion of a superior vertebral endplate the implant 120 is designed to contact when implanted at the first target location, and the second patient-specific topography or shape can match a topography of a portion of an inferior endplate that the implant 120 is designed to contact when implanted at the first target location. For example, the first surface 122 can have a patient-specific topography designed to match (e.g., mate with) the topography of the inferior endplate surface of the superior vertebral body to form a generally gapless interface therebetween. Likewise, the second surface 124 can have a patient-specific topography designed to match or mate with the topography of the superior endplate surface of the inferior vertebral body to form a generally gapless interface therebetween. As a result, the first and second surfaces 122, 124, can be irregularly contoured to match a contouring of the adjacent vertebral endplates. Also, as a result, the implant 120 can be asymmetrical with respect to a mid-sagittal plane of the implant 120 and/or with respect to a transverse plane of the implant 120. Other properties of the implant, including size, geometry, load-bearing characteristics, shear forces, etc., can also be patient-specific. Additional details of patient-specific implants, including designing and manufacturing patient specific implants, are described below in Section B. Further, additional examples of patient-specific implants that can used with the present technology are described in U.S. application Ser. Nos. 16/048,167, 16/242,877, 16/207,116, 16/352,699, 16/383,215, 16/569,494, 16/699,447, 16/735,222, 16/987,113, 16/990,810, 17/085,564, 17/100,396, 17/342,329, 17/518,524, 17/531,417, 17/835,777, 17/851,487, 17/867,621, and 17/842,242, each of which is incorporated by reference herein in its entirety.

The implant 120 can be a single, contiguous component (e.g., a one-piece or unitary interbody implant). For example, the implant 120 can be manufactured as a single structure using various additive manufacturing techniques, including extrusions, additive manufacturing processes, subtractive manufacturing processes, and/or other processes described throughout this Detailed Description. In some embodiments, the implant 120 is composed of metal (e.g., titanium, etc.) and/or a metal alloy (e.g., stainless steel, Nitinol, etc.). In other embodiments, the implant 120 can be composed of a biocompatible plastic. The implant 120 can also include a combination of lattice portions and solid portions. The combination of lattice portions and solid portions can be designed based on desired implant properties, such as stiffness, load-bearing capabilities, promotion of bone growth, fit, cost, or the like.

In embodiments in which the implant 120 is a patient-specific implant, it can be especially important to ensure the implant 120 is delivered to, and positioned at, the correct (e.g., intended) anatomical location within the patient to ensure maximum benefit from its patient specific design (e.g., at the first target location). For example, because the topography of the implant 120 is designed to mate with the topography of a specific portion of the adjacent vertebral bodies, placing the implant 120 offset from its intended target may result in reduced surface-to-surface contact between the implant 120 and the corresponding vertebral body, which may increase the likelihood of expulsion, subsidence, or other complications. As described in detail below, and without intending to be bound by theory, the positioning guide 130 is expected to assist with placing the implant 120 in the correct anatomical location.

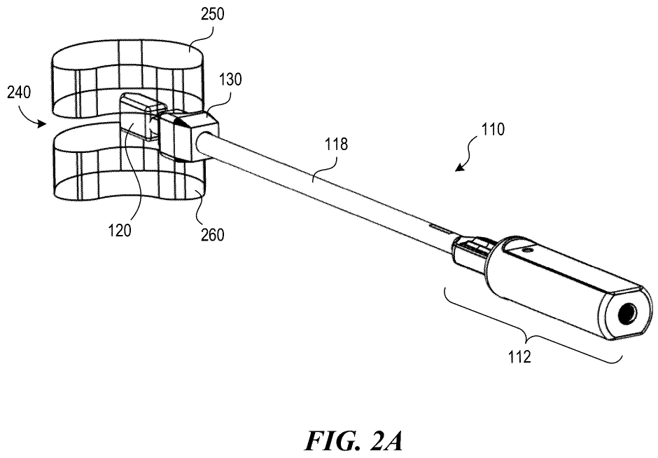

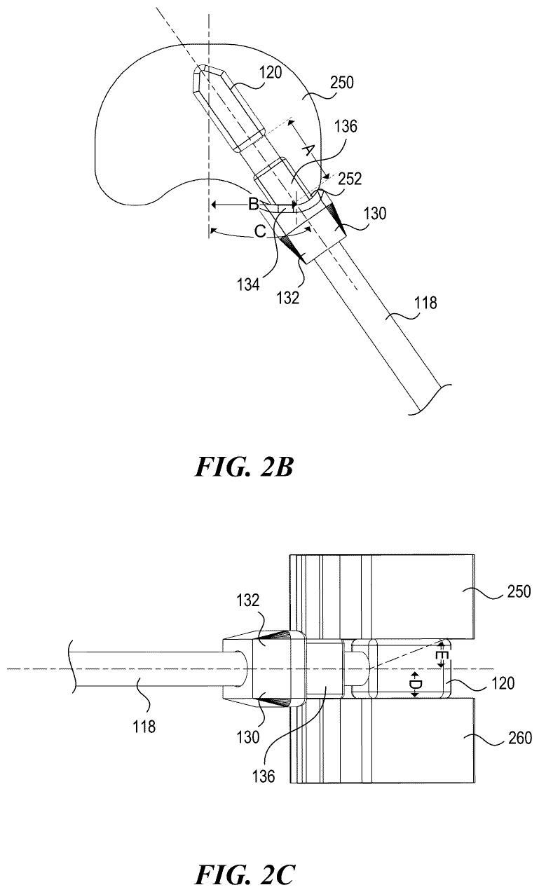

In particular, and as described in greater detail below with reference to FIGS. 1B and 1C, the positioning guide 130 can have one or more surfaces configured to mateably engage patient tissue at a second anatomical location, different than the first anatomical location, during the implant procedure. Accordingly, the positioning guide 130 can also be “patient-specific” in that it is designed to temporarily mate with one or more specific anatomical features of the patient (e.g., at the second target location). Of note, in some embodiments the positioning guide 130 and the implant 120 can have a predetermined spatial relationship therebetween that simultaneously permits the positioning guide 130 to occupy the second anatomical location and the implant 120 to occupy the first anatomical location. Indeed, as described in detail below with reference to FIGS. 2A-2C, positioning the positioning guide 130 at the second anatomical location can automatically cause the implant 120 to be placed at the first anatomical location. Thus, by placing the positioning guide 130 at the second target location before releasing the implant 120, a surgeon can have confidence that the implant 120 is positioned at the first target location, (e.g., without fluoroscopy or other visualization tools).

The positioning guide 130 can be coupled (e.g., releasably coupled) to the distal end 118b of the elongated shaft 118. This can be accomplished via a snap-fit, key-and-groove, or other suitable connection mechanism. In some embodiments, the positioning guide 130 can include one or more rotational clocking features, threads, or other features to ensure the positioning guide 130 is coupled to the elongated shaft 118 at the correct orientation. As described in greater detail below, the positioning guide 130 can also include a lumen or passage that is sized and shaped to permit the inner rod 119 to pass therethrough, such that the inner rod 119 can be releasably coupled to the implant 120, as described above. The inner rod 119 can be rotatable relative to the positioning guide 130, such that as a user rotates the dial 116 to disengage the inner rod 119 from the inserter instrument 110, the positioning guide 130 remains stationary.

FIGS. 1B and 1C, which are enlarged isometric views of the positioning guide 130 with other features of the system 100 omitted, illustrate additional features of the positioning guide 130. Referring first to FIG. 1B, the positioning guide 130 includes a base 132 and a depth stop 136 extending form the base 132. A distal portion 133 of the base 132 (e.g., the portion of the base facing away from the proximal complex 112 when coupled to the inserter instrument 110; FIG. 1A) can include contoured surfaces for engaging patient tissue. For example, in the illustrated embodiment the distal portion 133 of the base 132 includes a first contoured surface 134a, a second contoured surface 134b, a third contoured surface 134c, and a fourth contoured surface 134d (FIG. 1C) (referred to collectively as “the contoured surfaces 134”). The contoured surfaces 134 can be sized and shape to mate with a predetermined anatomical target (e.g., an anterior, posterior, or lateral margin of a vertebral endplate). Accordingly, as described in detail below, the contoured surfaces 134 can be “patient-specific surfaces” having a shape, size, and/or topography designed to match or otherwise mate with a patient-specific shape, size, and/or topography of the anatomical target.

The depth stop 136 can extend from the distal portion 133 of the base 132. The depth stop 136 can have a height H that is less than a distance between adjacent vertebral endplates (e.g., less than the intervertebral spacing) such that the depth stop 136 is sized and shaped to fit into intervertebral space. The depth stop 136 can also have a length L that defines a minimum distance between the distal portion 133 of the base 132 and the implant 120 (FIG. 1A). The length L of the depth stop 136 is defined such that the depth stop 136 causes the implant 120 to be positioned away from a margin of a vertebral body by at least the length L when the positioning guide 130 engages patient anatomy. The length L of the depth stop 136 can be patient-specific and predetermined in accordance with the first target location for the implant 120, and/or with the shape, size, and/or topography of the anatomical target.

In some embodiments, the contoured surfaces 134 and the length L of the depth stop 136 can be predetermined with reference to one or more surgical procedure techniques. The one or more surgical procedure techniques can include the first target location for the implant 120, a surgical path, a surgical access site, a surgeon's procedural preferences, and/or the like. In some embodiments, the contoured surfaces 134 and the length L of the depth stop 136 contact a generally large area of the vertebral body, such that the positioning guide 130 can provide angular guidance in addition to depth guidance. More specifically, the positioning guide 130 can control an angle (e.g., an axial angle relative to the midline of the patient, etc.) at which the implant is positioned in as it is driven forward, as described in greater detail below. In some embodiments, the positioning guide 130 can be used to insert and rotate the implant 120. For example, the positioning guide 130 can be designed to limit the rotation of the implant 120 to one direction and stop the implant 120 from rotating once it reaches its final orientation, (e.g., at the target implant location).

Referring collectively to FIGS. 1B and 1C, a lumen 138 can extend through the base 132 and the depth stop 136. The lumen 138 can be sized and shaped such that a portion of the inserter instrument can pass therethrough (e.g., the inner rod 119 of the inserter instrument 110; FIG. 1A). As set forth above, the lumen 138 can also be sized and shaped to permit the inner rod 119 to rotate freely relative to the lumen 138, which enables the inner rod 119 to be selectively disengaged from the implant 120 (FIG. 1A) once the implant 120 is positioned at the target anatomical location, e.g., to facilitate deployment of the implant 120. After deployment of the implant 120, the positioning guide 130 remains coupled to the inserter instrument.

As set forth above, the positioning guide 130 can be “patient-specific” in that it is specifically designed for use with a particular patient (e.g., to “match” patient anatomy at a target anatomical location). The positioning guide 130 can also be “implant-specific” in that it is specifically designed for use with a particular implant. That is, the positioning guide 130 can be specifically designed to assist with implanting a particular implant at a particular target anatomical location within a particular patient. Further, in some embodiments the spatial orientation and distance between the positioning guide 130 and the implant 120 can be fixed or at least stable. Thus, the positioning guide 130 can be designed such that when it occupies its target anatomical location (e.g., at a vertebral margin), the implant is positioned at its corresponding target anatomical location.

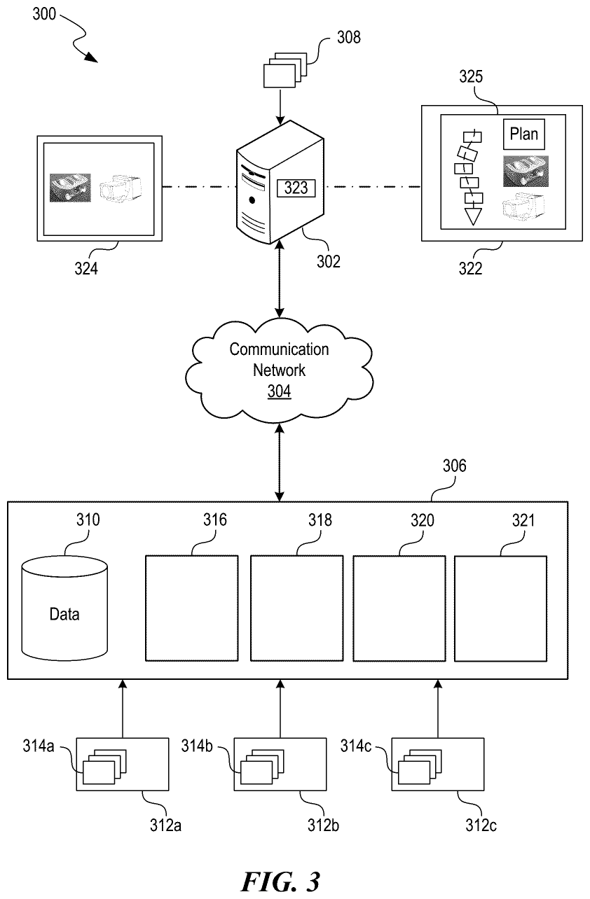

In some embodiments, the positioning guide 130 is composed of a plastic (e.g., biocompatible polymer) material. In other embodiments, the positioning guide 130 can be composed of stainless steel, titanium, or other metals or metal alloys. Regardless of its material composition, the positioning guide 130 can be a single, contiguous component (e.g., a one-piece or unitary component). As described in greater detail with reference to FIGS. 3-5, in some embodiments, the positioning guide 130 can be manufactured using additive manufacturing techniques (e.g., 3D printing). Because the positioning guide 130 is designed for use with implanting a particular implant at a particular location in a particular patient, the positioning guide 130 may be “single use.” Accordingly, it may be advantageous in certain embodiments to manufacture the positioning guide using relatively cheap techniques (e.g., 3D printing) and/or with relatively cheap materials (e.g., plastic).

FIGS. 2A-2C illustrate different views of a stage of a surgical procedure in which the inserter instrument of FIG. 1A is used deliver the implant 120 also of FIG. 1A into a patient's spine in accordance with embodiments of the present technology. Referring first to FIG. 2A, the inserter instrument 110 can be used to direct the implant 120 toward and into the first target location, which in the illustrated embodiment is an intervertebral space 240 between two adjacent endplates (e.g., a superior vertebral body 250 and an inferior vertebral body 260). In various embodiments, the superior vertebral body 250 and/or the inferior vertebral body 260 can be a cervical, thoracic, lumbar, or sacral vertebral body.

In some embodiments, a user can apply forces (e.g., via hammering, hand pressure, manual pushing, etc.) directly to the inserter instrument 110 to move the positioning guide 130 toward the second target location and the implant 120 toward and/or into the first target location. The force that moves the positioning guide 130 and the implant 120 can be proportional to the force applied directly to the proximal complex 112 of the inserter instrument 110. The proximal complex 112 can be, for example, moved side-to-side, rotated axially, pushed distally, and/or pulled proximally to position the implant 120 in the intervertebral space 240 at the first target location and the positioning guide 130 at the second target location. In addition, the inserter instrument 110 can include one or more clocked markings and/or handle orientation markers (not shown) to provide user feedback to ensure that implant 120 is inserted into the intervertebral space 240 at the correct orientation (e.g., the superior surface 122 (FIG. 1A) of the implant 120 faces an inferior endplate surface of the superior vertebral body 250 and the inferior surface 124 (FIG. 1A) of the implant 120 faces a superior endplate surface of the inferior vertebral body 260).

In some embodiments, the positioning guide 130 can be designed such that the implant 120 can be rotated in only a single direction, and up to a predetermined magnitude, once the implant 120 is in the intervertebral space. For example, axial rotation of the proximal complex 112 rotates the shaft 118 of the inserter instrument 110, thereby rotating the implant 120 in the intervertebral space 240. For example, the implant 120 can be rotated to a predetermined angle measurement relative to the endplates (e.g., 5 degrees, 15 degrees, 25 degrees, etc.).

FIG. 2B is an enlarged top view of the operative stage shown in FIG. 2A, with the proximal complex 112 and portions of the shaft 118 omitted for clarity. As shown, a user can advance the elongated shaft 118 until the positioning guide 130 contacts the vertebral bodies 250, 260. More specifically, the user can advance the elongated shaft 118 until the contoured surfaces 134 of the base 132 of the positioning guide 130 contact the anterior-lateral margin 252 of the vertebral bodies 250, 260. As described previously with reference to FIGS. 1B and 1C, the contoured surfaces 134 can be designed to match (e.g., mate with) the surface of the anterior-lateral margin 252 at the second target location. Accordingly, a user can manipulate the inserter instrument 110 (FIG. 2A) until the positioning guide 130 is positioned at the second target location. In some embodiments, a user can determine that the positioning guide 130 is positioned at the second target location based on tactile feedback, imaging (e.g., fluoroscopy), or other techniques. In some embodiments, the positioning guide 130 and/or inserter instrument 110 may include audible, tactile, or visual indicators that confirm when the positioning guide 130 is positioned at the second target location, and/or that confirm when the implant 120 is at the first target location. In some embodiments, the positioning guide 130 is designed to have a generally large area of contact between the contoured surfaces 134 and the anterior-lateral margin 252. In some embodiments, the generally large area of contact between the positioning guide 130 and the anterior-lateral margin 252 can precisely control angular guidance and/or depth guidance of the positioning guide 130 to the second target location, and/or the implant 120 to the first target location, such that resources necessary to confirm positioning (e.g., conventional surgical navigation systems, fluoroscopy, etc.) are reduced.

As described above, the positioning guide 130 and the implant 120 can have a predetermined spatial orientation relative to one another when carried by the inserter instrument 110. In particular, the predetermined spatial orientation can be set such that when the positioning guide 130 occupies the second target location at the anterior-lateral margin 252, the implant 120 is automatically positioned at the first target location in the intervertebral space 240 (FIG. 2A). In this way, positioning the positioning guide 130 at the second target location provides a user with confirmation that the implant 120 is positioned at the first target location.

For example, the positioning guide 130 can control/define the location of the implant 120 in multiple dimensions. The multiple dimensions can include at least three dimensions in the axial view: (1) a distance between the entry point and the implant 120, defined by a first length A, (2) a distance between the entry point and the midline, defined by a second length B, and (3) an axial angle C defined between the midline of the vertebral body and the longitudinal axis of the implant 120. The first length A can be determined by a length between the positioning guide 130 and the implant 120. The second length B and the axial angle C can be determined based on the position of the second target location at the anterior-lateral margin 252. In some embodiments, alternative landmarks (e.g., unique features of patient anatomy, previously implanted hardware, etc.) can be utilized as dimensions for controlling the implant location.

In some embodiments, the vertebral bodies 250, 260 may not have enough unique topographical features at the anterior-lateral margin 252 for the positioning guide 130 to precisely control one or more of the foregoing dimensions, such as distance between the entry point and the midline (the second length B) and/or the axial angle C. For example, in certain patients the anterior-lateral margin may have minimal grooves or other features that can be used to control angular guidance of the implant 120. In such embodiments, a user can rely on additional features in combination with the positioning guide 130 to ensure placement of the implant 120 at the first target location. For example, the elongated shaft 118 can include a level (e.g., a mechanical level, a digital level, etc.) to measure the angular position of implant 120 to determine the axial angle C. As another example, a user can manually or digitally measure the distance between the entry point and the midline (e.g., from a determined location on the shaft 118).

The positioning guide 130 can also control/define the location of the implant 120 in additional dimensions. For example, FIG. 2C is an enlarged lateral view of the operative stage shown in FIG. 2A, with the proximal complex 112 and portions of the shaft 118 omitted for clarity. As shown, the positioning guide 130 can control/define the location of the implant 120 in one or more dimensions in the lateral view. The one or more dimensions can include (1) a distance between the inferior vertebral body 260 and the implant center, defined by a third length D, and (2) a lateral angle E defined between a midline of the implant 120 relative to the inferior endplate of the superior vertebral body 250. Accordingly, the positioning guide 130 can ensure the implant 120 is deployed at the correct height from the inferior vertebral body 260 and at the correct lateral angle.

Once the implant 120 is positioned at the first target location, the inner rod 119 (FIG. 1A) can be unscrewed from the implant 120, and the inserter instrument 110 and the positioning guide 130 can be removed from the patient as described above, with respect to FIGS. 1A-1C. In some embodiments, the implant 120 is prevented from rotating due to its final placement between the two adjacent endplates (e.g., the superior vertebral body 250 and the inferior vertebral body 260). Therefore, the inner rod of the inserter instrument 110 rotates relative to the implant 120, disengaging the connection therebetween.

Without intending to be bound by theory, the positioning guide 130 is expected to improve the accuracy of placement of the implant 120 at the first target location, which as set forth above, is particularly important when implanting a patient-specific implant designed to occupy a specific location within the patient. In some embodiments, the positioning guide 130 can be used in place of a conventional surgical navigation system. In some embodiments, the positioning guide 130 may eliminate the need for, or at least reduce the use of, radiation (e.g., fluoroscopy) during a surgical procedure. The positioning guide 130 may also be cheaper than the foregoing navigation systems/techniques. Of course, the positioning guide 130 may provide additional advantages beyond those expressly recited herein.

In some embodiments, the positioning guide 130 and/or the inserter instrument 110 may include additional features beyond those described above that are expected to be useful with assisting with placement of the implant 120 at the first target location. As a first example, the positioning guide 130 and/or the inserter instrument 110 may include an angular measurement mechanism (not shown), e.g., to assist with controlling rotation of the implant 120 relative to the vertebral bodies (e.g., an axial angle of the implant). The angular measurement mechanism can be a mechanical level, a digital level, and/or other suitable instruments. As yet another example, in embodiments in which the implant 120 is used with fixation screws, spikes, or other fasteners, the positioning guide 130 can include corresponding fastener guides (e.g., bore holes, channels, etc.) for the screws, spikes, or other fasteners. The fastener guides can be designed such that the screws or other fasteners are inserted at a predetermined, custom trajectory. As yet another example, the inserter instrument 110 and/or positioning guide 130 can include two or more alignment features that can be aligned via line-of-sight (e.g., using fluoroscopy or the naked eye) to confirm accurate placement of the implant 120.

In some embodiments, the inserter instrument 110, the implant 120, and/or the positioning guide 130 can be used in combination with a two- or three-dimensions virtual anatomical model of the patient's spine with the implant 120. In such embodiments, the implant 120, the positioning guide 130, and/or the inserter instrument 110 can include radiographic markers that can be intra-operatively identified (e.g., via fluoroscopy) and compared to expected positions of the radiographic markers shown in the virtual model. This can provide additional confirmation that the implant 120 is positioned at the first anatomical location before deploying the implant 120. In such embodiments, images can be captured using an imaging device, such as a C-arm. Alternatively or additionally, the inserter instrument 110 and/or the positioning guide 130 may include an integrated camera that can provide real-time video and/or pictures of patient anatomy (e.g., vertebral bodies, intervertebral space, etc.), the implant 120, the positioning guide 130, and/or other aspects of the system 100. The integrated camera can assist a user in directing the positioning guide 130 toward the second target location, and/or confirm placement of the implant 120 at the first target location (e.g., by comparing the video or image data to the virtual model).

As one skilled in art will appreciate form the disclosure herein, the inserter instrument 110 and the positioning guide 130 can be readily adapted for use with various surgical procedures. For example, although largely described in the context of implanting a patient-specific implant, in some embodiments the inserter instrument 110 and positioning guide 130 can be used with a stock, or “off-the-shelf,” implant. Similarly, the positioning guide 130 can be used with a stock inserter instrument. The positioning guide 130 can also be utilized for k-wire based procedures.

In some embodiments, the system 100 can include two or more positioning guides 130. For example, a procedure for implanting a TLIF-C implant can be performed in two stages, with each stage having a different positioning guide. The first stage can include using a first positioning guide to position the TLIF-C implant straight at a first angle and a first depth relative to an insertion point. A second positioning guide can then pivot the TLIF-C implant to a second angle and a second depth relative to the first positioning guide. The pivoting motion of the TLIF-C implant is defined by point-to-point translation of the posterior pivot point on the TLIF-C implant and/or the inserter instrument 110.

### B. SYSTEMS AND METHODS FOR DESIGNING PATIENT-SPECIFIC POSITIONING GUIDES AND PATIENT-SPECIFIC IMPLANTS

FIG. 3 is a network connection diagram illustrating a system 300 for providing patient-specific medical care, according to embodiments of the present technology. As described in further detail herein, the system 300 is configured to generate a medical treatment plan for a patient. In some embodiments, the system 300 is configured to generate a medical treatment plan for a patient suffering from an orthopedic or spinal disease or disorder, such as trauma (e.g., fractures), cancer, deformity, degeneration, pain (e.g., back pain, leg pain), irregular spinal curvature (e.g., scoliosis, lordosis, kyphosis), irregular spinal displacement (e.g., spondylolisthesis, lateral displacement axial displacement), osteoarthritis, lumbar degenerative disc disease, cervical degenerative disc disease, lumbar spinal stenosis, or cervical spinal stenosis, or a combination thereof. The medical treatment plan can include surgical information, technology recommendations (e.g., device and/or instrument recommendations), and/or medical device designs. For example, the medical treatment plan can include at least surgical procedure (e.g., a surgical procedure or intervention), at least one medical device (e.g., an implanted medical device such as the implant 120 of FIGS. 1A-2C), and at least one positioning guide (e.g., the positioning guide 130 of FIGS. 1A-2C). In some embodiments, the medical treatment plan is therefore also referred to as a “surgical plan,” a “patient-specific surgical plan,” a “patient-specific treatment plan,” or the like.

In some embodiments, the system 300 generates a medical treatment plan that is customized for a particular patient or group of patients, also referred to herein as a “patient-specific” or “personalized” treatment or surgical plan. The patient-specific surgical plan can include at least one patient-specific surgical procedure and/or at least one patient-specific medical device that are designed and/or optimized for the patient's particular characteristics (e.g., condition, anatomy, pathology, condition, medical history). For example, the patient-specific medical device can be designed and manufactured specifically for the particular patient, rather than being an off-the-shelf device. However, it shall be appreciated that a patient-specific surgical plan can also include aspects that are not customized for the particular patient. For example, a patient-specific or personalized surgical procedure can include one or more instructions, portions, steps, etc. that are non-patient-specific. Likewise, a patient-specific or personalized medical device can include one or more components that are non-patient-specific, and/or can be used with an instrument or tool that is non-patient-specific. Personalized implant designs can be used to manufacture or select patient-specific technologies, including medical devices, instruments, and/or surgical kits. For example, a personalized surgical kit can include one or more patient-specific devices (e.g., implants, positioning guides, etc.), patient-specific instruments, non-patient-specific technology (e.g., standard instruments, devices, etc.), instructions for use, patient-specific treatment plan information, or a combination thereof.

The system 300 includes a client computing device 302, which can be a user device, such as a smart phone, mobile device, laptop, desktop, personal computer, tablet, phablet, or other such devices known in the art. As discussed further herein, the client computing device 302 can include one or more processors, and memory storing instructions executable by the one or more processors to perform the methods described herein. The client computing device 302 can be associated with a healthcare provider (e.g., a surgeon, healthcare administrator, hospital system, etc.) that is treating the patient. Although FIG. 3 illustrates a single client computing device 302, in alternative embodiments, the client computing device 302 can instead be implemented as a client computing system encompassing a plurality of computing devices, such that the operations described herein with respect to the client computing device 302 can instead be performed by the client computing system and/or the plurality of client computing devices.

The client computing device 302 is configured to receive a patient data set 308 associated with a patient to be treated. The patient data set 308 can include data representative of the patient's condition, anatomy, pathology, medical history, preferences, and/or any other information or parameters relevant to the patient. For example, the patient data set 308 can include medical history, surgical intervention data, treatment outcome data, progress data (e.g., physician notes), patient feedback (e.g., feedback acquired using quality of life questionnaires, surveys), clinical data, provider information (e.g., physician, hospital, surgical team), patient information (e.g., demographics, sex, age, height, weight, type of pathology, occupation, activity level, tissue information, health rating, comorbidities, health related quality of life (HRQL)), vital signs, diagnostic results, medication information, allergies, image data (e.g., camera images, Magnetic Resonance Imaging (MRI) images, ultrasound images, Computerized Aided Tomography (CAT) scan images, Positron Emission Tomography (PET) images, X-Ray images), diagnostic equipment information (e.g., manufacturer, model number, specifications, user-selected settings/configurations, etc.), or the like. In some embodiments, the patient data set 308 includes data representing one or more of patient identification number (ID), age, gender, body mass index (BMI), lumbar lordosis, Cobb angle(s), pelvic incidence, disc height, segment flexibility, bone quality, rotational displacement, and/or treatment level of the spine.

The client computing device 302 is also configured to enable a user (e.g., a surgeon) to review one or more proposed surgical plans for a patient to be treated. In particular, the client computing device 302 can include a surgical plan review software module 323 (“the review module 323”). The review module 323 can comprise computer-executable instructions for generating, displaying, and/or implementing a surgical plan review program or platform 325 (“the review program 325”) that facilitates surgeon or user review of one or more patient-specific surgical plans via the client computing device 302. As described in greater detail below, a surgeon or other user can review, modify, and/or approve a surgical plan, a three-dimensional model of patient anatomy, a predicted post-operative three-dimensional model of patient anatomy, an implant design, and/or a positioning guide design using the review program 325.

The review module 323 can be stored in the form of computer-readable or computer-executable instructions on a memory (not shown) of the client computing device 302. In other embodiments, the review module 323 can be stored remotely from the client computing device 302 (e.g., in the cloud or at a remote server) and implemented on the client computing device 302 via a remote (e.g., wireless) connection. In yet other embodiments, some of the review module 323 can be stored locally at the client computing device 302 while other aspects of the review module 323 can be store remotely.

The client computing device 302 is operably connected via a communication network 304 to a server 306, thus allowing for data transfer between the client computing device 302 and the server 306. The communication network 304 may be a wired and/or a wireless network. The communication network 304, if wireless, may be implemented using communication techniques such as Visible Light Communication (VLC), Worldwide Interoperability for Microwave Access (WiMAX), Long term evolution (LTE), Wireless local area network (WLAN), Infrared (IR) communication, Public Switched Telephone Network (PSTN), Radio waves, and/or other communication techniques known in the art.

The server 306, which may also be referred to as a “treatment assistance network” or “prescriptive analytics network,” can include one or more computing devices and/or systems. As discussed further herein, the server 306 can include one or more processors, and memory storing instructions executable by the one or more processors to perform some or all of the methods described herein. In some embodiments, the server 306 is implemented as a distributed “cloud” computing system or facility across any suitable combination of hardware and/or virtual computing resources.

The client computing device 302 and the server 306 can individually or collectively perform some or all of the various methods described herein for providing patient-specific medical care. For example, some or all of the steps of the methods described herein can be performed by the client computing device 302 alone, the server 306 alone, or a combination of the client computing device 302 and the server 306. Thus, although certain operations are described herein with respect to the server 306, it shall be appreciated that these operations can also be performed by the client computing device 302, and vice-versa, unless the context clearly dictates otherwise.

The server 306 includes at least one database 310 configured to store reference data useful for the treatment planning methods described herein. The reference data can include historical and/or clinical data from the same or other patients, data collected from prior surgeries and/or other treatments of patients by the same or other healthcare providers, data relating to medical device designs, data relating to positioning guide designs, data collected from study groups or research groups, data from practice databases, data from academic institutions, data from implant manufacturers or other medical device manufacturers, data from imaging studies, data from simulations, clinical trials, demographic data, treatment data, outcome data, mortality rates, or the like.

In some embodiments, the database 310 includes a plurality of reference patient data sets, each patient reference data set associated with a corresponding reference patient. For example, the reference patient can be a patient that previously received treatment or is currently receiving treatment. Each reference patient data set can include data representative of the corresponding reference patient's condition, anatomy, pathology, medical history, disease progression, preferences, and/or any other information or parameters relevant to the reference patient, such as any of the data described herein with respect to the patient data set 308. In some embodiments, the reference patient data set includes pre-operative data, intra-operative data, and/or post-operative data. For example, a reference patient data set can include data representing one or more of patient ID, age, gender, BMI, lumbar lordosis, Cobb angle(s), pelvic incidence, disc height, segment flexibility, bone quality, rotational displacement, and/or treatment level of the spine. As another example, a reference patient data set can include treatment data regarding at least one surgical procedure performed on the reference patient, such as descriptions of surgical procedures or interventions (e.g., surgical approaches, bony resections, surgical maneuvers, corrective maneuvers, placement of implants or other devices). In some embodiments, the treatment data includes medical device design data for at least one medical device used to treat the reference patient, such as physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties). In some embodiments, the treatment data includes positioning guide design data for at least one positioning guide used to implant the medical device within the reference patient. In yet another example, a reference patient data set can include outcome data representing an outcome of the treatment of the reference patient, such as corrected anatomical metrics, presence of fusion, HRQL, pain level, activity level, return to work, complications, recovery times, efficacy, mortality, and/or follow-up surgeries.

In some embodiments, the server 306 receives at least some of the reference patient data sets from a plurality of healthcare provider computing systems (e.g., systems 312a-312c, collectively “healthcare provider computing systems 312”). The server 306 can be connected to the healthcare provider computing systems 312 via one or more communication networks (not shown). Each healthcare provider computing system 312 can be associated with a corresponding healthcare provider (e.g., physician, surgeon, medical clinic, hospital, healthcare network, etc.). Each healthcare provider computing system 312 can include at least one reference patient data set (e.g., reference patient data sets 314a-314c, collectively “reference patient data sets 314”) associated with reference patients treated by the corresponding healthcare provider. The reference patient data sets 314 can include, for example, electronic medical records, electronic health records, biomedical data sets, etc. The reference patient data sets 314 can be received by the server 306 from the healthcare provider computing systems 312 and can be reformatted into different formats for storage in the database 310. Optionally, the reference patient data sets 314 can be processed (e.g., cleaned) to ensure that the represented patient parameters are likely to be useful in the treatment planning methods described herein.

As described in further detail herein, the server 306 can be configured with one or more algorithms that generate patient-specific surgical plan data (e.g., treatment procedures, target anatomical corrections, medical devices, positioning guides, etc.) based on the reference data. In some embodiments, the patient-specific data is generated based on correlations between the patient data set 308 and the reference data. Optionally, the server 306 can predict outcomes, including recovery times, efficacy based on clinical end points, likelihood of success, predicted mortality, predicted related follow-up surgeries, or the like. In some embodiments, the server 306 can continuously or periodically analyze patient data (including patient data obtained during the patient stay) to determine near real-time or real-time risk scores, mortality prediction, etc.

In some embodiments, the server 306 includes one or more modules for performing one or more steps of the patient-specific treatment planning methods described herein. For example, in the depicted embodiment, the server 306 includes a data analysis module 316, a treatment planning module 318, a disease progression module 320, and an intervention timing module 321. In alternative embodiments, one or more of these modules may be combined with each other, or may be omitted. Thus, although certain operations are described herein with respect to a particular module or modules, this is not intended to be limiting, and such operations can be performed by a different module or modules in alternative embodiments.

The data analysis module 316 is configured with one or more algorithms for identifying a subset of reference data from the database 310 that is likely to be useful in developing a patient-specific treatment plan. For example, the data analysis module 316 can compare patient-specific data (e.g., the patient data set 308 received from the client computing device 302) to the reference data from the database 310 (e.g., the reference patient data sets) to identify similar data (e.g., one or more similar patient data sets in the reference patient data sets). The comparison can be based on one or more parameters, such as age, gender, BMI, lumbar lordosis, pelvic incidence, and/or treatment levels. The parameter(s) can be used to calculate a similarity score for each reference patient. The similarity score can represent a statistical correlation between the patient data set 308 and the reference patient data set. Accordingly, similar patients can be identified based on whether the similarity score is above, below, or at a specified threshold value. For example, as described in greater detail below, the comparison can be performed by assigning values to each parameter and determining the aggregate difference between the subject patient and each reference patient. Reference patients whose aggregate difference is below a threshold can be considered to be similar patients.

The data analysis module 316 can further be configured with one or more algorithms to select a subset of the reference patient data sets, e.g., based on similarity to the patient data set 308 and/or treatment outcome of the corresponding reference patient. For example, the data analysis module 316 can identify one or more similar patient data sets in the reference patient data sets, and then select a subset of the similar patient data sets based on whether the similar patient data set includes data indicative of a favorable or desired treatment outcome. The outcome data can include data representing one or more outcome parameters, such as corrected anatomical metrics, presence of fusion, HRQL, activity level, complications, recovery times, efficacy, mortality, or follow-up surgeries. As described in further detail below, in some embodiments, the data analysis module 316 calculates an outcome score by assigning values to each outcome parameter. A patient can be considered to have a favorable outcome if the outcome score is above, below, or at a specified threshold value.

In some embodiments, the data analysis module 316 selects a subset of the reference patient data sets based at least in part on user input (e.g., from a clinician, surgeon, physician, healthcare provider). For example, the user input can be used in identifying similar patient data sets. In some embodiments, weighting of similarity and/or outcome parameters can be selected by a healthcare provider or physician to adjust the similarity and/or outcome score based on clinician input. In further embodiments, the healthcare provider or physician can select the set of similarity and/or outcome parameters (or define new similarity and/or outcome parameters) used to generate the similarity and/or outcome score, respectively.

In some embodiments, the data analysis module 316 includes one or more algorithms used to select a set or subset of the reference patient data sets based on criteria other than patient parameters. For example, the one or more algorithms can be used to select the subset based on healthcare provider parameters (e.g., based on healthcare provider ranking/scores such as hospital/physician expertise, number of procedures performed, hospital ranking, etc.) and/or healthcare resource parameters (e.g., diagnostic equipment, facilities, surgical equipment such as surgical robots), or other non-patient related information that can be used to predict outcomes and risk profiles for procedures for the present healthcare provider. For example, reference patient data sets with images captured from similar diagnostic equipment can be aggregated to reduce or limit irregularities due to variation between diagnostic equipment. Additionally, patient-specific treatment plans can be developed for a particular health-care provider using data from similar healthcare providers (e.g., healthcare providers with traditionally similar outcomes, physician expertise, surgical teams, etc.). In some embodiments, reference healthcare provider data sets, hospital data sets, physician data sets, surgical team data sets, post-treatment data set, and other data sets can be utilized. By way of example, a patient-specific surgical plan to perform a battlefield surgery can be based on reference patient data from similar battlefield surgeries and/or data sets associated with battlefield surgeries. In another example, the patient-specific surgical plan can be generated based on available robotic surgical systems. The reference patient data sets can be selected based on patients that have been operated on using comparable robotic surgical systems under similar conditions (e.g., size and capabilities of surgical teams, hospital resources, etc.).

The treatment planning module 318 is configured with one or more algorithms to generate at least one surgical plan (e.g., pre-operative plans, intra-operative plans, post-operative plans etc.) based on the output from the data analysis module 316. In some embodiments, the treatment planning module 318 is configured to develop and/or implement at least one predictive model for generating the patient-specific treatment plan, also known as a “prescriptive model.” The predictive model(s) can be developed using clinical knowledge, statistics, machine learning, AI, neural networks, or the like. In some embodiments, the output from the data analysis module 316 is analyzed (e.g., using statistics, machine learning, neural networks, AI) to identify correlations between data sets, patient parameters, healthcare provider parameters, healthcare resource parameters, treatment procedures, medical device designs, positioning guide designs, and/or treatment outcomes. These correlations can be used to develop at least one predictive model that predicts the likelihood that a surgical plan will produce a favorable outcome for the particular patient. The predictive model(s) can be validated, e.g., by inputting data into the model(s) and comparing the output of the model to the expected output.

In some embodiments, the treatment planning module 318 is configured to generate the surgical plan based on previous treatment data from reference patients. For example, the treatment planning module 318 can receive a selected subset of reference patient data sets and/or similar patient data sets from the data analysis module 316 and determine or identify treatment data from the selected subset. The treatment data can include, for example, treatment procedure data (e.g., surgical procedure or intervention data), medical device design data (e.g., implant design data), and/or positioning guide design data that are associated with favorable or desired treatment outcomes for the corresponding patient. The treatment planning module 318 can analyze the treatment procedure data, medical device design data, and/or positioning guide design data to determine an optimal treatment protocol for the patient to be treated. For example, the treatment procedures, medical device designs, and positioning guide designs can be assigned values and aggregated to produce a treatment score. The patient-specific surgical plan can be determined by selecting surgical plan(s) based on the score (e.g., higher or highest score; lower or lowest score; score that is above, below, or at a specified threshold value). The personalized patient-specific surgical plan can be based on, at least in part, the patient-specific technologies or patient-specific selected technology.

Alternatively, or in combination, the treatment planning module 318 can generate the surgical plan based on correlations between data sets. For example, the treatment planning module 318 can correlate treatment procedure data, medical device design data, and/or positioning guide design data from similar patients with favorable outcomes (e.g., as identified by the data analysis module 316). Correlation analysis can include transforming correlation coefficient values to values or scores. The values/scores can be aggregated, filtered, or otherwise analyzed to determine one or more statistical significances. These correlations can be used to determine surgical procedure(s), medical device design(s), and/or positioning guide design(s) that are optimal or likely to produce a favorable outcome for the patient to be treated.

Alternatively, or in combination, the treatment planning module 318 can generate the surgical plan using one or more AI techniques. AI techniques can be used to develop computing systems capable of simulating aspects of human intelligence, e.g., learning, reasoning, planning, problem solving, decision making, etc. AI techniques can include, but are not limited to, case-based reasoning, rule-based systems, artificial neural networks, decision trees, support vector machines, regression analysis, Bayesian networks (e.g., naïve Bayes classifiers), genetic algorithms, cellular automata, fuzzy logic systems, multi-agent systems, swarm intelligence, data mining, machine learning (e.g., supervised learning, unsupervised learning, reinforcement learning), and hybrid systems.

In some embodiments, the treatment planning module 318 generates the surgical plan using one or more trained machine learning models. Various types of machine learning models, algorithms, and techniques are suitable for use with the present technology. In some embodiments, the machine learning model is initially trained on a training data set, which is a set of examples used to fit the parameters (e.g., weights of connections between “neurons” in artificial neural networks) of the model. For example, the training data set can include any of the reference data stored in database 310, such as a plurality of reference patient data sets or a selected subset thereof (e.g., a plurality of similar patient data sets).

In some embodiments, the machine learning model (e.g., a neural network or a naïve Bayes classifier) may be trained on the training data set using a supervised learning method (e.g., gradient descent or stochastic gradient descent). The training data set can include pairs of generated “input vectors” with the associated corresponding “answer vector” (commonly denoted as the target). The current model is run with the training data set and produces a result, which is then compared with the target, for each input vector in the training data set. Based on the result of the comparison and the specific learning algorithm being used, the parameters of the model are adjusted. The model fitting can include both variable selection and parameter estimation. The fitted model can be used to predict the responses for the observations in a second data set called the validation data set. The validation data set can provide an unbiased evaluation of a model fit on the training data set while tuning the model parameters. Validation data sets can be used for regularization by early stopping, e.g., by stopping training when the error on the validation data set increases, as this may be a sign of overfitting to the training data set. In some embodiments, the error of the validation data set error can fluctuate during training, such that ad-hoc rules may be used to decide when overfitting has truly begun. Finally, a test data set can be used to provide an unbiased evaluation of a final model fit on the training data set.

To generate a surgical plan, the patient data set 308 can be input into the trained machine learning model(s). Additional data, such as the selected subset of reference patient data sets and/or similar patient data sets, and/or treatment data from the selected subset, can also be input into the trained machine learning model(s). The trained machine learning model(s) can then calculate whether various candidate treatment procedures, medical device designs, and/or positioning guide designs are likely to produce a favorable outcome for the patient. Based on these calculations, the trained machine learning model(s) can select at least one surgical plan for the patient. In some embodiments, the trained machine learning model(s) can determine candidate procedures (or candidate surgical plans), analyze the candidate procedures, select the candidate surgical plans or portions thereof, score plans, and/or generate surgical plans for the patient. Each surgical plan can be scored (e.g., scored based on favorable outcome, likelihood of outcome, etc.) and ranked according to the score. The trained machine learning model(s) can determine a set of surgical plans that meet selection criteria for plan review by a user. The selection criteria can be based on, for example, regulatory requirements, reimbursement criteria, healthcare/provider expertise, available surgical equipment, manufacturing capabilities, elimination criteria, combinations thereof, or the like. A user can input one or more selection criteria to control the types and/or features of the surgical plans for comparison. In embodiments where multiple trained machine learning models are used, the models can be run sequentially or concurrently to compare outcomes and can be periodically updated using training data sets. The treatment planning module 318 can use one or more of the machine learning models based the model's predicted accuracy score.

The patient-specific surgical plan generated by the treatment planning module 318 can include at least one patient-specific surgical procedure (e.g., a surgical procedure or intervention), at least one patient-specific medical device (e.g., an implant or implant delivery instrument), and at least one patient-specific positioning guide. A patient-specific surgical plan can include an entire surgical procedure or portions thereof. Additionally, one or more patient-specific medical devices can be specifically selected or designed for the corresponding surgical procedure, thus allowing for the various components of the patient-specific technology to be used in combination to treat the patient.

In some embodiments, the patient-specific surgical procedure includes an orthopedic surgery procedure, such as spinal surgery, hip surgery, knee surgery, jaw surgery, hand surgery, shoulder surgery, elbow surgery, total joint reconstruction (arthroplasty), skull reconstruction, foot surgery, or ankle surgery. Spinal surgery can include spinal fusion surgery, such as posterior lumbar interbody fusion (PLIF), anterior lumbar interbody fusion (ALIF), transverse or transforaminal lumbar interbody fusion (TLIF), lateral lumbar interbody fusion (LLIF), direct lateral lumbar interbody fusion (DLIF), or extreme lateral lumbar interbody fusion (XLIF). In some embodiments, the patient-specific treatment procedure includes descriptions of and/or instructions for performing one or more aspects of a patient-specific surgical procedure. For example, the patient-specific surgical procedure can include one or more of a surgical approach, a corrective maneuver, a bony resection, or implant placement.

In some embodiments, the patient-specific medical device design includes a design for an orthopedic implant and/or a design for an instrument for delivering an orthopedic implant. Examples of such implants include, but are not limited to, screws (e.g., bone screws, spinal screws, pedicle screws, facet screws), interbody implant devices (e.g., intervertebral implants), cages, plates, rods, disks, fusion devices, spacers, rods, expandable devices, stents, brackets, ties, scaffolds, fixation device, anchors, nuts, bolts, rivets, connectors, tethers, fasteners, joint replacements, hip implants, or the like. Examples of instruments include, but are not limited to, screw guides, cannulas, ports, catheters, insertion tools, removal tools, awls, drivers, or the like.

A patient-specific medical device design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of a corresponding medical device. For example, a design for an orthopedic implant can include implant shape, size, material, and/or effective stiffness (e.g., lattice density, number of struts, location of struts, etc.). In some embodiments, the generated patient-specific medical device design is a design for an entire device. Alternatively, the generated design can be for one or more components of a device, rather than the entire device.

In some embodiments, the design is for one or more patient-specific device components that can be used with standard, off-the-shelf components. For example, in a spinal surgery, an ALIF kit can include both standard components and patient-specific customized components. In some embodiments, the ALIF kit can include a patient specific interbody device that can be used with standard interfixating screws. In some embodiments, the generated design is for a patient-specific implant that can be used with a standard, off-the-shelf delivery instrument. For example, the implants (e.g., interbody device, screws, screw holders, rods) can be designed and manufactured for the patient, while the instruments for delivering the implants can be standard instruments. This approach allows the components that are implanted to be designed and manufactured based on the patient's anatomy and/or surgeon's preferences to enhance treatment. The patient-specific devices described herein are expected to improve delivery into the patient's body, placement at the treatment site, and/or interaction with the patient's anatomy.

A patient-specific positioning guide design can include data representing one or more of size, shape, surface topology, angular orientation, surface area for tissue contact, and/or other features or metrics described above with reference to FIGS. 1A-2C. In some embodiments, the generated patient-specific positioning guide design is a design for the entire positioning guide. Alternatively, the generated design can be for one or more components of the guide (e.g., the contoured surfaces 134; FIG. 1B), rather than the entire guide.

In embodiments in which the patient-specific surgical plan includes a specific surgical procedure to implant a medical device, the treatment planning module 318 can also store various types of implant surgery information, such as implant parameters (e.g., types, dimensions), availability of implants, aspects of a pre-operative plan (e.g., initial implant configuration, detection and measurement of the patient's anatomy, etc.), FDA requirements for implants (e.g., specific implant parameters and/or characteristics for compliance with FDA regulations), or the like. In some embodiments, the treatment planning module 318 can convert the implant surgery information into formats useable for machine-learning based models and algorithms. For example, the implant surgery information can be tagged with particular identifiers for formulas or can be converted into numerical representations suitable for supplying to the trained machine learning model(s). The treatment planning module 318 can also store information regarding the patient's anatomy, such as two- or three-dimensional images or models of the anatomy, and/or information regarding the biology, geometry, and/or mechanical properties of the anatomy. The anatomy information can be used to inform implant design and/or placement.

The disease progression module 320 can be used to analyze, predict, and/or model disease progression for a particular patient. In particular, the disease progression module 320 can estimate the rate of disease progression for the patient under a variety of different circumstances, including (a) if no surgical intervention occurs, and (b) if one or more surgical plans (e.g., surgical procedures identified by the treatment planning module 318) are performed. The disease progression module 320 can therefore include an algorithm, machine learning model, or other software analytical tool for predicting disease progression in a particular patient. The surgical treatment plans, patient-specific implants, and/or positioning guides described herein can therefore be at least partially based on the estimated rates of disease progression, enabling the modeling of different outcomes over a desired period of times. Accordingly, the present technology can generate one or more virtual simulations of the predicted disease progression to demonstrate how the patient's anatomy is predicted to change over time. Physician input can be used to generate or modify the virtual simulation(s). The present technology can generate one or more post-treatment virtual simulations based on the received physician input for review by the healthcare provider, patient, etc.

Based off the modeled disease progression, the systems and methods described herein can also (i) identify a recommended time for surgical intervention, (ii) identify a recommended type of surgical procedure for the patient, (iii) simulate changes to patient anatomy, and/or (iv) identify expected changes in patient anatomy that may increase or decrease the utility of utilizing a positioning guide to implant a medical device. In some embodiments, the present technology therefore includes an intervention timing module 321 that includes an algorithm, machine learning model, or other software analytical tool for determining the optimal time for surgical intervention in a particular patient. This can be done, for example, by analyzing patient reference data that includes (i) pre-operative disease progression metrics for individual reference patients, (ii) disease metrics at the time of surgical intervention for individual reference patients, (iii) post-operative disease progression metrics for individual reference patients, and/or (iv) scored surgical outcomes for individual reference patients. The intervention timing module 321 can compare the disease metrics for a particular patient to the reference patient data sets to determine, for similar patients, the point of disease progression at which surgical intervention produced the most favorable outcomes.

Without being bound by theory, incorporating disease progression modeling into the patient-specific surgical plans described herein may even further increase the effectiveness of the procedures and/or provide a surgeon more data by which to evaluate various surgical plans. For example, in many cases it may be disadvantageous to operate after a patient's disease progresses to an irreversible or unstable state. However, it may also be disadvantageous to operate too early, such as before the patient's disease is causing symptoms and/or if the patient's disease may not progress further. The disease progression module 320 and/or the intervention timing module 321 can therefore help identify the window of time during which surgical intervention in a particular patient has the highest probability of providing a favorable outcome for the patient.

The surgical plan(s) generated by the treatment planning module 318 can be transmitted via the communication network 304 to the client computing device 302 for output to a user (e.g., clinician, surgeon, healthcare provider, patient). In some embodiments, the client computing device 302 includes or is operably coupled to a display 322 for outputting the treatment plan(s). The display 322 can include a graphical user interface (GUI) for visually depicting various aspects of the surgical plan(s). For example, the display 322 can show various aspects of a surgical procedure to be performed on the patient, such as the surgical approach, treatment levels, corrective maneuvers, tissue resection, and/or implant placement. To facilitate visualization, the surgical plan can include a virtual model of the surgical procedure that can be displayed via the display 322. The display 322 may also display additional aspects of the surgical plan, such as predicted post-operative patient metrics, predicted disease progression metrics associated with the identified surgical procedure, etc. As another example, the display 322 can show a design for a medical device to be implanted in the patient in accordance with the transmitted surgical plan, such as a two- or three-dimensional model of the device design. The display 322 can also show a design for the positioning guide to be used to implant the medical device. The display 322 can also show patient information, such as two- or three-dimensional images or models of the patient's anatomy where the surgical procedure is to be performed and/or where the device is to be implanted. The client computing device 302 can further include one or more user input devices (not shown) allowing the user to modify, select, approve, and/or reject the displayed treatment plan(s).

In some embodiments, one or more aspects of the surgical plan are displayed using the review program 325. For example, the review program 325, which may be implemented as a mobile phone application, a computer application, or the like, can display (e.g., via the display 322) one or more aspects of the surgical plan (e.g., a surgical procedure, a virtual model of patient anatomy, an implant, a positioning guide, etc.). The review program 325 may provide an interactive interface that further enables a surgeon to select between different patients, select between different surgical plans for the same patient, compare surgical plans for the same patient, review the status of a surgical plan, provide feedback on a proposed surgical plan, accept a surgical plan, reject a surgical plan, etc. The review program 325 may further enable a surgeon or other user to select between different views of a virtual model of patient anatomy, and/or different views of a patient-specific implant and/or positioning guide to be used in the surgical plan.

In some embodiments, the medical device design(s) and the positioning guide design(s) generated by the treatment planning module 318 can be transmitted from the client computing device 302 and/or server 306 to a manufacturing system 324 for manufacturing a corresponding medical device and/or positioning guide. The manufacturing system 324 can be located on site or off site. On-site manufacturing can reduce the number of sessions with a patient and/or the time to be able to perform the surgery whereas off-site manufacturing can be useful make the complex devices. Off-site manufacturing facilities can have specialized manufacturing equipment. In some embodiments, more complicated device components can be manufactured off site, while simpler device components can be manufactured on site.

Various types of manufacturing systems are suitable for use in accordance with the embodiments herein. For example, the manufacturing system 324 can be configured for additive manufacturing, such as three-dimensional (3D) printing, stereolithography (SLA), digital light processing (DLP), fused deposition modeling (FDM), selective laser sintering (SLS), selective laser melting (SLM), selective heat sintering (SHM), electronic beam melting (EBM), laminated object manufacturing (LOM), powder bed printing (PP), thermoplastic printing, direct material deposition (DMD), inkjet photo resin printing, or like technologies, or combination thereof. Alternatively, or in combination, the manufacturing system 324 can be configured for subtractive (traditional) manufacturing, such as CNC machining, electrical discharge machining (EDM), grinding, laser cutting, water jet machining, manual machining (e.g., milling, lathe/turning), or like technologies, or combinations thereof. The manufacturing system 324 can manufacture one or more patient-specific medical devices and/or positioning guides based on fabrication instructions or data (e.g., CAD data, 3D data, digital blueprints, stereolithography data, or other data suitable for the various manufacturing technologies described herein). Different components of the system 300 can generate at least a portion of the manufacturing data used by the manufacturing system 324. The manufacturing data can include, without limitation, fabrication instructions (e.g., programs executable by additive manufacturing equipment, subtractive manufacturing equipment, etc.), 3D data, CAD data (e.g., CAD files), CAM data (e.g., CAM files), path data (e.g., print head paths, tool paths, etc.), material data, tolerance data, surface finish data (e.g., surface roughness data), regulatory data (e.g., FDA requirements, reimbursement data, etc.), or the like. The manufacturing system 324 can analyze the manufacturability of the implant design and/or positioning guide design based on the received manufacturing data. The implant and/or positioning guide design can be finalized by altering geometries, surfaces, etc. and then generating manufacturing instructions. In some embodiments, the server 306 generates at least a portion of the manufacturing data, which is transmitted to the manufacturing system 324.

The manufacturing system 324 can generate CAM data, print data (e.g., powder bed print data, thermoplastic print data, photo resin data, etc.), or the like and can include additive manufacturing equipment, subtractive manufacturing equipment, thermal processing equipment, or the like. The additive manufacturing equipment can be 3D printers, stereolithography devices, digital light processing devices, fused deposition modeling devices, selective laser sintering devices, selective laser melting devices, electronic beam melting devices, laminated object manufacturing devices, powder bed printers, thermoplastic printers, direct material deposition devices, or inkjet photo resin printers, or like technologies. The subtractive manufacturing equipment can be CNC machines, electrical discharge machines, grinders, laser cutters, water jet machines, manual machines (e.g., milling machines, lathes, etc.), or like technologies. Both additive and subtractive techniques can be used to produce implants with complex geometries, surface finishes, material properties, etc. The generated fabrication instructions can be configured to cause the manufacturing system 324 to manufacture the patient-specific orthopedic implant and/or positioning guide that matches or is therapeutically the same as the patient-specific design. In some embodiments, the patient-specific medical device and/or positioning guide can include features, materials, and designs shared across designs to simplify manufacturing. For example, deployable patient-specific medical devices for different patients can have similar internal deployment mechanisms but have different deployed configurations. In some embodiments, the components of the patient-specific medical devices are selected from a set of available pre-fabricated components and the selected pre-fabricated components can be modified based on the fabrication instructions or data.

The surgical plans described herein can be performed by a surgeon, a surgical robot, or a combination thereof, thus allowing for treatment flexibility. In some embodiments, the surgical procedure can be performed entirely by a surgeon, entirely by a surgical robot, or a combination thereof. For example, one step of a surgical procedure can be manually performed by a surgeon and another step of the procedure can be performed by a surgical robot. In some embodiments the treatment planning module 318 generates control instructions configured to cause a surgical robot (e.g., robotic surgery systems, navigation systems, etc.) to perform a surgical procedure partially or fully. The control instructions can be transmitted to the robotic apparatus by the client computing device 302 and/or the server 306.

Following the treatment of the patient in accordance with the surgical plan, treatment progress can be monitored over one or more time periods to update the data analysis module 316, treatment planning module 318, disease progression module 320, and/or intervention timing module 321. Post-treatment data can be added to the reference data stored in the database 310. The post-treatment data can be used to train machine learning models for developing patient-specific treatment plans, patient-specific medical devices, or combinations thereof.

It shall be appreciated that the components of the system 300 can be configured in many different ways. For example, in alternative embodiments, the database 310, the data analysis module 316, the treatment planning module 318, the disease progression module 320, and/or the intervention timing module 321 can be components of the client computing device 302, rather than the server 306. As another example, the database 310, the data analysis module 316, the treatment planning module 318, the disease progression module 320, and/or the intervention timing module 321 can be located across a plurality of different servers, computing systems, or other types of cloud-computing resources, rather than at a single server 306 or client computing device 302.

Additionally, in some embodiments, the system 300 can be operational with numerous other computing system environments or configurations. Examples of computing systems, environments, and/or configurations that may be suitable for use with the technology include, but are not limited to, personal computers, server computers, handheld or laptop devices, cellular telephones, wearable electronics, tablet devices, multiprocessor systems, microprocessor-based systems, programmable consumer electronics, network PCs, minicomputers, mainframe computers, distributed computing environments that include any of the above systems or devices, or the like.

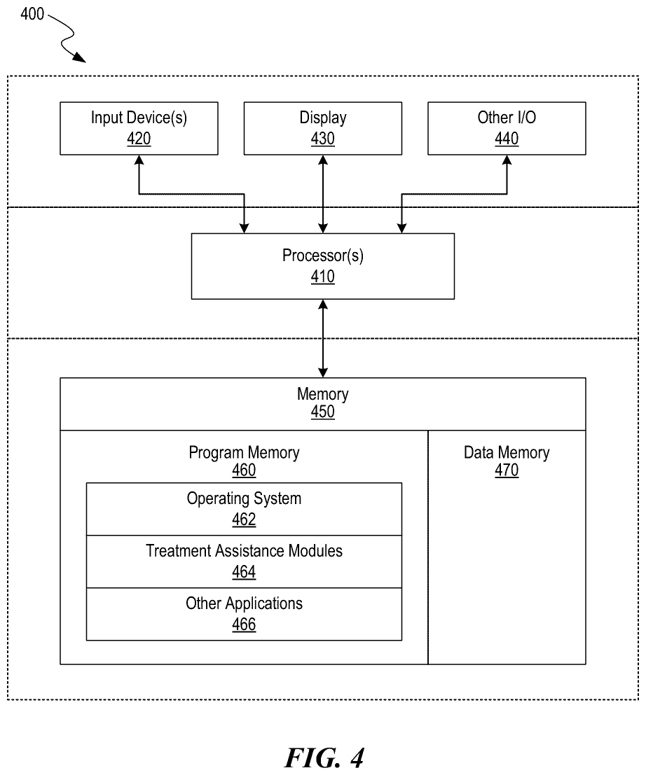

FIG. 4 illustrates a computing device 400 suitable for use in connection with the system 300 of FIG. 3, according to an embodiment. The computing device 400 can be incorporated in various components of the system 300 of FIG. 3, such as the client computing device 302 or the server 306. The computing device 400 includes one or more processors 410 (e.g., CPU(s), GPU(s), HPU(s), etc.). The processor(s) 410 can be a single processing unit or multiple processing units in a device or distributed across multiple devices. The processor(s) 410 can be coupled to other hardware devices, for example, with the use of a bus, such as a PCI bus or SCSI bus. The processor(s) 410 can be configured to execute one more computer-readable program instructions, such as program instructions to carry out of any of the methods described herein.

The computing device 400 can include one or more input devices 420 that provide input to the processor(s) 410, e.g., to notify it of actions from a user of the computing device 400. The actions can be mediated by a hardware controller that interprets the signals received from the input device and communicates the information to the processor(s) 410 using a communication protocol. Input device(s) 420 can include, for example, a mouse, a keyboard, a touchscreen, an infrared sensor, a touchpad, a wearable input device, a camera- or image-based input device, a microphone, or other user input devices.

The computing device 400 can include a display 430 used to display various types of output, such as text, models, virtual procedures, surgical plans, implants, graphics, and/or images (e.g., images with voxels indicating radiodensity units or Hounsfield units representing the density of the tissue at a location). In some embodiments, the display 430 provides graphical and textual visual feedback to a user. The processor(s) 410 can communicate with the display 430 via a hardware controller for devices. In some embodiments, the display 430 includes the input device(s) 420 as part of the display 430, such as when the input device(s) 420 include a touchscreen or is equipped with an eye direction monitoring system. In alternative embodiments, the display 430 is separate from the input device(s) 420. Examples of display devices include an LCD display screen, an LED display screen, a projected, holographic, or augmented reality display (e.g., a heads-up display device or a head-mounted device), and so on.

Optionally, other I/O devices 440 can also be coupled to the processor(s) 410, such as a network card, video card, audio card, USB, firewire or other external device, camera, printer, speakers, CD-ROM drive, DVD drive, disk drive, or Blu-Ray device. Other I/O devices 440 can also include input ports for information from directly connected medical equipment such as imaging apparatuses, including MRI machines, X-Ray machines, CT machines, etc. Other I/O devices 440 can further include input ports for receiving data from these types of machines from other sources, such as across a network or from previously captured data, for example, stored in a database.

In some embodiments, the computing device 400 also includes a communication device (not shown) capable of communicating wirelessly or wire-based with a network node. The communication device can communicate with another device or a server through a network using, for example, TCP/IP protocols. The computing device 400 can utilize the communication device to distribute operations across multiple network devices, including imaging equipment, manufacturing equipment, etc.

The computing device 400 can include memory 450, which can be in a single device or distributed across multiple devices. Memory 450 includes one or more of various hardware devices for volatile and non-volatile storage and can include both read-only and writable memory. For example, a memory can comprise random access memory (RAM), various caches, CPU registers, read-only memory (ROM), and writable non-volatile memory, such as flash memory, hard drives, floppy disks, CDs, DVDs, magnetic storage devices, tape drives, device buffers, and so forth. A memory is not a propagating signal divorced from underlying hardware; a memory is thus non-transitory. In some embodiments, the memory 450 is a non-transitory computer-readable storage medium that stores, for example, programs, software, data, or the like. In some embodiments, memory 450 can include program memory 460 that stores programs and software, such as an operating system 462, one or more treatment assistance modules 464, and other application programs 466. The treatment assistance module(s) 464 can include one or more modules configured to perform the various methods described herein (e.g., the data analysis module 316 and/or treatment planning module 318 described with respect to FIG. 3). Memory 450 can also include data memory 470 that can include, e.g., reference data, configuration data, settings, user options or preferences, etc., which can be provided to the program memory 460 or any other element of the computing device 400.

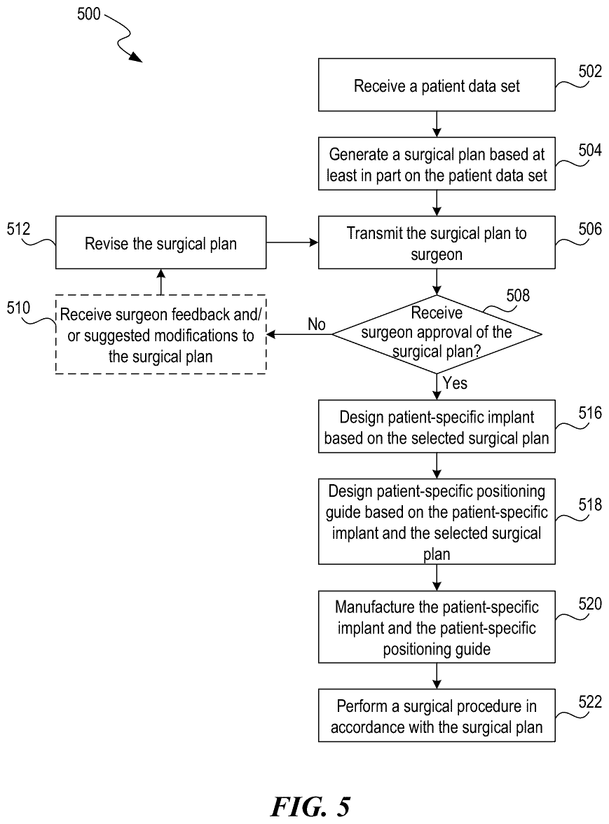

FIG. 5 is a flow diagram illustrating a method 500 for providing patient-specific medical care, according to an embodiment of the present technology. Some or all of the method 500 can be performed by various computing systems or software modules, including, for example, the computing systems described above with respect to FIGS. 3 and 4.

The method 500 can begin at block 502 by receiving a patient data set for a particular patient in need of medical treatment. The patient data set can include data representative of the patient's condition, anatomy, pathology, symptoms, medical history, preferences, and/or any other information or parameters relevant to the patient. For example, the patient data set can include surgical intervention data, treatment outcome data, progress data (e.g., surgeon notes, etc.), patient feedback (e.g., feedback acquired using quality of life questionnaires, surveys, etc.), clinical data, patient information (e.g., demographics, sex, age, height, weight, type of pathology, occupation, activity level, tissue information, health rating, comorbidities, health related quality of life (HRQL), etc.), vital signs, diagnostic results, medication information, allergies, diagnostic equipment information (e.g., manufacturer, model number, specifications, user-selected settings/configurations, etc.) or the like. The patient data set can also include image data, such as camera images, Magnetic Resonance Imaging (MRI) images, ultrasound images, Computerized Aided Tomography (CAT) scan images, Positron Emission Tomography (PET) images, X-Ray images, and the like. In some embodiments, the patient data set includes data representing one or more of patient identification number (ID), age, gender, body mass index (BMI), lumbar lordosis, Cobb angle(s), pelvic incidence, disc height, segment flexibility, bone quality, rotational displacement, and/or treatment level of the spine. The patient data set can be received at a server, computing device, or other computing system. For example, in some embodiments the patient data set can be received by the server 306 shown in FIG. 3. In some embodiments, the computing system that receives the patient data set at block 502 also stores one or more software modules (e.g., the data analysis module 316, the treatment planning module 318, the disease progression module 320, and/or the intervention timing module 321, shown in FIG. 3, or additional software modules for performing various operations of the method 500).

In some embodiments, the received patient data set can include disease metrics such as lumbar lordosis, Cobb angles, coronal parameters (e.g., coronal balance, global coronal balance, coronal pelvic tilt, etc.), sagittal parameters (e.g., pelvic incidence, sacral slope, thoracic kyphosis, etc.) and/or pelvic parameters. The disease metrics can include micro-measurements (e.g., metrics associated with specific or individual segments of the patient's spine) and/or macro-measurements (e.g., metrics associated with multiple segments of the patient's spine). In some embodiments, the disease metrics are not included in the patient data set, and the method 500 includes determining (e.g., automatically determining) one or more of the disease metrics based on the patient image data, as described below. In some embodiments, the received patient data can include functional mobility test scores (e.g., step test, six-meter walk test, sit-to-stand test, timed up and go test, etc.). The received patient data set can include additional subjective test scores that reflect aspects of the patient condition, such as pain tests (e.g., Visual Analog Scale (VAS) pain scores, Low Back Pain Rating scale scores, etc.), disability tests (e.g., Oswestry Disability Index scores, Quebec back pain disability test scores, etc.), quality of life tests (e.g., Quality of Life Scale scores), etc.

The method 500 can continue at block 504 by generating a surgical plan based at least in part on the patient data set received at block 502. As described in detail below, the surgical plan can include a target location or region of interest for surgical intervention and one or more surgical procedures or interventions to be performed at the region of interest. The surgical plan can also include predicted post-operative data associated with performing the surgical procedure at the target location. For example, the surgical plan may include a predicted or target post-operative anatomical configuration shown as a two- or three-dimensional virtual model. In some embodiments, the surgical plan also includes additional predicted post-operative analytics, such as predicted disease progression, predicted patient satisfaction, predicted patient mobility, predicted patient pain, predicted patient quality of life, etc.

In some embodiments, the operation of generating the surgical plan includes identifying a specific target location to be involved in the surgical procedure. For example, in the context of spinal fusion surgery, generating the surgical plan may include identifying one or more vertebral levels for fusion. In some embodiments, the vertebral level is a lumbar vertebral level (e.g., L1-L5) and/or the sacrum. In some embodiments, the identified target location includes a specific range of vertebral levels to be involved in a surgery (e.g., L5-S1, L3-L4, etc.). The identified target location may include two, three, four, five, or more vertebral levels. Of course, the foregoing target locations are provided by way of example only, and the present technology is not limited to the anatomical locations listed above. Indeed, in some embodiments the target location may include other vertebral levels, such as cervical and/or thoracic vertebral levels, and/or anatomical structures other than the spine, such as the hip, knee, ankle, shoulder, elbow, wrist, hand, the jaw, the skull, or other anatomical locations, as described throughout this Detailed Description.

The target location can be identified by reviewing image data of the patient. In some embodiments, a computing system (e.g., the server 306 of FIG. 3) and/or one or more software modules (e.g., the treatment planning module 318 of FIG. 3) can review and analyze patient image data and automatically identify the target location. In such embodiments, a trained machine learning program or other software-based program can analyze patient image data, extract measurements from the patient image data, compare the extracted measurements to reference data (e.g., predetermined thresholds or ranges associated with “healthy” patients normalized for age, sex, gender, etc.), and identify anatomical regions that are candidates for surgical correction. Alternatively, or additionally, the target location can be identified and/or confirmed through other suitable means, such as via a technician or healthcare provider reviewing image data and identifying anatomical deformities.

As provided above, in some embodiments the operation of generating the surgical plan also includes identifying a surgical procedure for the patient. In embodiments in which the surgical plan includes identifying a target location, the surgical procedure can be associated with the target location. In the context of spinal surgery, representative surgical procedures include spinal fusion, artificial disc replacement, vertebroplasty, kyphoplasty, spinal laminectomy/decompression, discectomy, facetectomy, foraminotomy, or other spine surgery procedures. Examples of spinal fusion surgery include posterior lumbar interbody fusion (PLIF), anterior lumbar interbody fusion (ALIF), transverse or transforaminal lumbar interbody fusion (TLIF), lateral lumbar interbody fusion (LLIF), direct lateral lumbar interbody fusion (DLIF), or extreme lateral lumbar interbody fusion (XLIF). The foregoing spinal fusion surgeries are provided by way of example only, and the present technology can include identifying any type of spinal or other surgical procedures at block 504.

The surgical procedure associated with the surgical plan can be identified using any of the methods and systems described herein. For example, in some embodiments the server 306 of FIG. 3 and/or the associated software modules (e.g., the treatment planning module 318) can identify one or more surgical procedures based on, for example, user input, the received or extracted patient data, and/or the identified target location(s). For example, if the server 306 determines that a patient is suffering from disc degeneration from L3-L5, the server 306 may recommend an ALIF procedure to fuse L2-T12. Alternatively, the server 306 may recommend an artificial disc replacement at L3-L4 and L4-L5 to correct the degeneration while preserving motion. The surgical procedure can be identified using other methods and systems as well.

In some embodiments, the operation at block 504 can include reviewing and/or analyzing multiple types of surgical procedures and/or surgical steps to identify the surgical procedure for inclusion within the surgical plan. Types of surgical procedures and/or surgical steps can be selected for inclusion with the surgical plan (or eliminated from inclusion with the surgical plan) based on, for example, user input, insurance coverage of the procedure or step, healthcare provider parameters (e.g., based on healthcare provider ranking/scores such as hospital/physician expertise, number of similar procedures performed, hospital ranking for procedure, etc.), healthcare resource parameters (e.g., diagnostic equipment, facilities, surgical equipment such as surgical robots), and/or other non-patient related information (e.g., information that can be used to score, predict outcomes and risk profiles for procedures for the present healthcare provider, and/or rank procedures).

In some embodiments, the operation of generating the surgical plan includes identifying or designing a corrected anatomical configuration for the patient (the corrected anatomical configuration can also be referred to herein as the “planned configuration,” “optimized geometry,” “post-operative anatomical configuration,” or “target outcome”). The corrected anatomical configuration can reflect the desired and/or predicted anatomy of the patient if the surgical plan were performed. In some embodiments, generating the surgical plan includes generating one or more virtual models (two-dimensional models, three-dimensional models, etc.) showing the corrected anatomical configuration. The virtual model may include some or all of the patient's anatomy within the target location (e.g., any combination of tissue types including, but not limited to, bony structures, cartilage, soft tissue, vascular tissue, nervous tissue, etc.). In some embodiments, the corrected anatomical configuration is identified/determined before the surgical procedure and/or target location. That is, a computing system or user can model a preferred anatomical outcome, and based on the desired anatomical outcome, identify a surgical procedure and target location that will achieve the desired anatomical outcome once performed.

In some embodiments, generating the surgical plan includes generating one or more patient metrics associated with the corrected anatomical configuration. In the context of spinal surgery, patient metrics may include, for example, coronal parameters, sagittal parameters, pelvic parameters, Cobb angles, shoulder tilt, iliolumbar angles, coronal balance, lordosis angles, intervertebral space height, or other similar spinal parameters. Similar as described above, the patient metrics can be determined before identifying a surgical procedure and/or target location for surgical intervention. That is, a computing system or user can use the patient metrics to identify a surgical procedure and target location that will achieve the patient metrics once performed.

The surgical plan can include additional features. In some embodiments, for example, the surgical plan can include predicted disease progression, predicted patient satisfaction, predicted patient mobility, predicted patient pain, predicted patient quality of life, or the like. For example, the surgical plan may include estimates of disease progression if the patient were to undergo the identified surgical procedure at the identified target location. That is, the surgical plan can include virtual models (e.g., two-dimensional or three-dimensional virtual models) of patient anatomy at various intervals post-operation. For example, the surgical plan may include a predictive model of patient anatomy at one or more of 6 months post-op, 1 year post-op, 2 years post-op, 3 years post-op, 4 years post-op, 5 years post-op, 6 years post-op, 7 years post-op, 8 years post-op, 9 years post-op, and/or 10 years post-op. The disease progression model may also include predicted patient metrics (e.g., any of the patient metrics described herein, including coronal parameters, sagittal parameters, pelvic parameters, Cobb angles, shoulder tilt, iliolumbar angles, coronal balance, lordosis angles, intervertebral space height, or other similar spinal parameters) at any of the various post-operative intervals identified above, in addition to or in lieu of including the virtual model of predicted patient anatomy.

Once generated, the surgical plan can be digitally displayed as a surgical report on one or more display screens for ease of review, editing, annotation, the like. In some embodiments, the surgical plan can be stored as computer-executable instructions that can be executed via the review module 323 on the client computing device 302 of FIG. 3.

In some embodiments, the operation of generating the surgical plan at block 504 includes generating a plurality of candidate surgical plans (or subsets of surgical plans such as surgical procedures), and then selecting the surgical from within the plurality of candidate surgical plans. For example, in some embodiments a computing system can automatically identify a plurality (e.g., two, three, four, five, six, seven, eight, nine, ten, or more) of surgical plans (or subsets of surgical plans such as surgical procedures) based on the patient-data set and/or one or more user-inputted criteria. The identified candidate surgical plans may be ranked and/or scored based on various factors, including predicted patient outcomes, user-review, etc. The highest ranked identified candidate surgical plans (e.g., based on predicted patient outcomes) can be selected as the surgical plan. In some embodiments, certain ranked surgical plans may not be selected as the surgical plan based on user review and/or failure to meet various user criteria. For example, if a particular surgical plan is identified as requiring a surgical procedure that the physician is unfamiliar with, the particular surgical plan may not be selected, and the method can instead include selecting the next best surgical plan as the surgical plan. Accordingly, in some implementations, physician-specific scoring is used to score candidate procedures/surgical plans before selecting the surgical plan. For example, procedures with scores meeting a threshold score (e.g., threshold post-operative metrics score, physician inputted threshold score, threshold outcome score, etc.) can be identified for user review. The system can therefore compare advantages and disadvantages of candidate procedures with respect to each other before selecting the surgical plan.

Once the surgical plan is generated at block 504, the method 500 can continue at block 506 by transmitting the surgical plan to a surgeon. In some embodiments, the same computing system used at blocks 502 and 504 can transmit the surgical plan to a computing device for surgeon review (e.g., the client computing device 302 described in FIG. 3). This can include directly transmitting the surgical plan to the computing device or uploading the first and second surgical plans to a cloud or other storage system for subsequent downloading.

The surgeon can review the surgical plan and, at block 508, approve or disapprove of the surgical plan. For example, the surgeon may review the surgical plan using the review program 325 (FIG. 3) to determine whether the surgeon deems the surgical plan acceptable. This may include, for example, reviewing the surgical plans target locations, surgical procedure, target/predicted post-operative anatomical configuration, and predicted post-operative patient metrics.

In some embodiments, the surgeon may not approve the surgical plan at block 508. In such embodiments, the surgeon can optionally provide feedback and/or suggested modifications to the surgical plan (e.g., by adjusting the virtual model or changing one or more aspects about the plan, providing comments on or more requested changes to the surgical plan, etc.). Accordingly, the method 500 can optionally include receiving (e.g., via the computing system) the surgeon feedback and/or suggested modifications at block 510. This may include, for example, modifying target locations for surgical intervention, surgical procedures, and/or target post-operative anatomical configuration. If surgeon feedback and/or suggested modifications are received at block 510, the method 500 can continue at block 512 by revising (e.g., automatically revising via the computing system) the surgical plan based at least in part on the surgeon feedback and/or suggested modifications received at block 510. In some embodiments, the surgeon does not provide feedback and/or suggested modifications if they reject the surgical plan. In such embodiments, block 510 can be omitted, and the method 500 can continue at block 512 by revising (e.g., automatically revising via the computing system) the surgical plans by selecting new and/or additional reference patient data sets and/or generating a new candidate surgical plan. The revised and/or new surgical plan can then be transmitted to the surgeon for review. The operations at blocks 506, 508, 510, and 512 can be repeated as many times as necessary until the surgeon selects and approves a particular surgical plan.

Once surgeon approval of a surgical plan is received at block 508, the method 500 can continue at block 516 by designing (e.g., via the same computing system that performed blocks 502-508) a patient-specific implant based on the selected surgical plan. For example, the patient-specific implant can be designed based on the target location and surgical procedure included in the selected surgical plan. The patient-specific implant(s) can also be specifically designed such that, when implanted in the particular patient at the target location using the identified surgical procedure, it directs the patient's anatomy to occupy the target post-operative anatomical configuration (e.g., transforming the patient's anatomy from the patient's native anatomical configuration to the corrected anatomical configuration). The patient-specific fusion implant can be designed such that, when implanted, it causes the patient's anatomy to occupy the corrected anatomical configuration for the expected service life of the implant (e.g., 5 years or more, 10 years or more, 20 years or more, 50 years or more, etc.). In some embodiments, the patient-specific fusion implant is designed solely based on the virtual model of the corrected anatomical configuration and/or without reference to pre-operative patient images.

The patient-specific fusion implant can be any of the implants described herein. In other embodiments, the patient-specific fusion implant can include other implants, such as those described in U.S. application Ser. Nos. 16/048,167, 16/242,877, 16/207,316, 16/352,699, 16/383,215, 16/569,494, 16/699,447, 16/735,222, 16/987,113, 16/990,810, 17/085,564, 17/300,396, 17/342,329, 17/518,524, 17/531,417, 17/835,777, 17/851,487, 17/867,621, and 17/842,242, each of which is incorporated by reference herein in its entirety. The patient-specific implant design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of the implant. For example, a design for an orthopedic implant can include implant shape, size, material, and/or effective stiffness (e.g., lattice density, number of struts, location of struts, etc.). In addition to the interbody device, in some embodiments the patient-specific fusion implant can further include one or more screws (e.g., bone screws, spinal screws, pedicle screws, facet screws), cages, plates, rods, discs, spacers, expandable devices, stents, brackets, ties, scaffolds, fixation device, anchors, nuts, bolts, rivets, connectors, tethers, fasteners, or the like.

In some embodiments, designing the implant at block 516 can optionally include generating fabrication instructions for manufacturing the implant. For example, the computing system may generate computer-executable fabrication instructions that that, when executed by a manufacturing system, cause the manufacturing system to manufacture the implant.

In some embodiments, the patient-specific implant is designed at block 516 only after the surgeon has selected a surgical plan. Accordingly, in some embodiments, the implant design is neither transmitted to the surgeon with the surgical plan at block 508, nor manufactured before receiving surgeon approval of the surgical plan. Without being bound by theory, waiting to design the patient-specific implant until after the surgeon approves the surgical plan may increase the efficiency of the method 500 and/or reduce the resources necessary to perform the method 500. In other embodiments, one or more patient-specific implants can be designed and included in the surgical plans transmitted to the surgeon at block 506. For example, a virtual implant of the patient-specific implant can be generated and transmitted for surgeon review concurrent with the surgical plan during the operation of block 506. Accordingly, in some embodiments the operation at block 514 can be included within the block 504.

During and/or after designing the implant at block 516, the method 500 can continue at block 518 by designing (e.g., via the same computing system that performed blocks 502-508) a patient-specific positioning guide based on the selected surgical plan and the patient-specific implant. For example, the patient-specific positioning guide can be designed based on the target implant location for the patient-specific implant, the surgical approach (e.g., lateral vs. anterior vs. posterior), unique anatomical features of the patient (e.g., as shown in the patient data set and/or the virtual model), or the like. More specifically, the patient-specific positioning guide can be designed to specifically mate with patient anatomy at a second target location, as described previously with reference to FIGS. 1A-2C. The patient-specific positioning guide can further be designed to control one or more location parameters of the patient-specific implant, including, but not limited to, a distance between an entry point and the implant, a distance between an entry point and the vertebral body midline, an angle between the entry point and the vertebral body midline, a distance between the vertebral body implant and the implant midline, a lateral angle of the implant, an axial angle of the implant, and/or other parameters associated with a position of the implant.

In some embodiments, designing the positioning guide at block 518 can optionally include generating fabrication instructions for manufacturing the positioning guide. For example, the computing system may generate computer-executable fabrication instructions that that, when executed by a manufacturing system, cause the manufacturing system to manufacture the positioning guide.

In some embodiments, and similar to the implant, the patient-specific positioning guide is designed at block 518 only after the surgeon has selected a surgical plan. Accordingly, in some embodiments, the positioning guide design is neither transmitted to the surgeon with the surgical plan at block 508, nor manufactured before receiving surgeon approval of the surgical plan. Without being bound by theory, waiting to design the patient-specific positioning guide until after the surgeon approves the surgical plan may increase the efficiency of the method 500 and/or reduce the resources necessary to perform the method 500. In other embodiments, one or more patient-specific positioning guides can be designed and included in the surgical plans transmitted to the surgeon at block 506. For example, a virtual rendering of the positioning guide can be generated and transmitted for surgeon review concurrent with the surgical plan during the operation of block 506. Accordingly, in some embodiments the operation at block 518 can be included within the block 504.

The method 500 can continue at block 520 by manufacturing the patient-specific implant and the patient-specific positioning guide. The implant and/or the positioning guide can be manufactured using additive manufacturing techniques, such as 3D printing, stereolithography, digital light processing, fused deposition modeling, selective laser sintering, selective laser melting, electronic beam melting, laminated object manufacturing, powder bed printing, thermoplastic printing, direct material deposition, or inkjet photo resin printing, or like technologies, or combination thereof. Alternatively, or additionally, the implant and/or the positioning guide can be manufactured using subtractive manufacturing techniques, such as CNC machining, electrical discharge machining (EDM), grinding, laser cutting, water jet machining, manual machining (e.g., milling, lathe/turning), or like technologies, or combinations thereof. The implant and/or the positioning guide can be manufactured using the same or different manufacturing techniques and/or materials. For example, in some embodiments a first manufacturing technique (e.g., 3D printing) can be used to manufacture the patient-specific positioning guide, whereas a second manufacturing technique (e.g., CNC machining) can be used to manufacture the patient-specific implant. In this way, the manufacturing technique can be optimized based on the material composition, complexity, necessary life-span, and/or desired cost of the patient-specific implant versus the patient-specific positioning guide. Furthermore, the patient-specific positioning guide can be manufactured with a first, relatively cheaper material (e.g., plastic), and the patient-specific implant can be manufactured with a second, relatively more durable, material, such as stainless steel, titanium, etc. The implant and/or positioning guide may be manufactured by any suitable manufacturing system (e.g., the manufacturing system 324 shown in FIG. 3). In some embodiments, the implant and the positioning guide are manufactured by the manufacturing system executing the computer-readable fabrication instructions generated by the computing system at blocks 516 and 518, respectively.

Once the implant and positioning guide are manufactured at block 520, the method 500 can continue at block 522 by performing the selected surgical plan and implanting the patient-specific implant into the patient. This can include, for example, using the patient-specific positioning guide to ensure the patient-specific implant is positioned at its intended location in the patient. The patient-specific positioning guide remains coupled to the inserter instrument following implantation of the patient-specific interbody implant, such that the patient-specific positioning guide is removed from the patient with the inserter instrument. Aspects of the surgical plan, such as some or all of the surgical procedure, can be performed manually, by a robotic surgical platform (e.g., a surgical robot), or a combination thereof. In embodiments in which the surgical procedure is performed at least in part by a robotic surgical platform, the surgical plan can include computer-readable control instructions configured to cause the surgical robot to perform, at least partly, the patient-specific surgical procedure.

The method 500 can be implemented and performed in various ways. In some embodiments, the operations at blocks 502-518 can be performed by a computing system associated with a first entity, block 520 can be performed by a manufacturing system associated with a second entity, and block 522 can be performed by a surgical provider, surgeon, and/or robotic surgical platform associated with a third entity. Any of the foregoing blocks may also be implemented as computer-readable instructions stored in memory and executable by one or more processors of the associated computing system(s).

### C. CONCLUSION

As one skilled in the art will appreciate, any of the software modules described previously may be combined into a single software module for performing the operations described herein. Likewise, the software modules can be distributed across any combination of the computing systems and devices described herein, and are not limited to the express arrangements described herein. Accordingly, any of the operations described herein can be performed by any of the computing devices or systems described herein, unless expressly noted otherwise.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In some embodiments, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” or “operably coupled,” to each other to achieve the desired functionality, and any two components capable of being so associated can also be viewed as being “operably couplable” to each other to achieve the desired functionality. Specific examples of operably couplable include but are not limited to physically mateable and/or physically interacting components and/or wirelessly interactable and/or wirelessly interacting components and/or logically interacting and/or logically interactable components.

The embodiments, features, systems, devices, materials, methods, and techniques described herein may, in some embodiments, be similar to any one or more of the embodiments, features, systems, devices, materials, methods and techniques described in the following:
U.S. application Ser. No. 16/048,167, filed on Jul. 27, 2018, titled “SYSTEMS AND METHODS FOR ASSISTING AND AUGMENTING SURGICAL PROCEDURES;”
U.S. application Ser. No. 16/242,877, filed on Jan. 8, 2019, titled “SYSTEMS AND METHODS OF ASSISTING A SURGEON WITH SCREW PLACEMENT DURING SPINAL SURGERY;”
U.S. application Ser. No. 16/207,116, filed on Dec. 1, 2018, titled “SYSTEMS AND METHODS FOR MULTI-PLANAR ORTHOPEDIC ALIGNMENT;”
U.S. application Ser. No. 16/352,699, filed on Mar. 13, 2019, titled “SYSTEMS AND METHODS FOR ORTHOPEDIC IMPLANT FIXATION;”
U.S. application Ser. No. 16/383,215, filed on Apr. 12, 2019, titled “SYSTEMS AND METHODS FOR ORTHOPEDIC IMPLANT FIXATION;”
U.S. application Ser. No. 16/569,494, filed on Sep. 12, 2019, titled “SYSTEMS AND METHODS FOR ORTHOPEDIC IMPLANTS;” and
U.S. application Ser. No. 16/699,447, filed Nov. 29, 2019, titled “SYSTEMS AND METHODS FOR ORTHOPEDIC IMPLANTS;”
U.S. application Ser. No. 16/735,222, filed Jan. 6, 2020, titled “PATIENT-SPECIFIC MEDICAL PROCEDURES AND DEVICES, AND ASSOCIATED SYSTEMS AND METHODS;”
U.S. application Ser. No. 16/987,113, filed Aug. 6, 2020, titled “PATIENT-SPECIFIC ARTIFICIAL DISCS, IMPLANTS AND ASSOCIATED SYSTEMS AND METHODS;”
U.S. application Ser. No. 16/990,810, filed Aug. 11, 2020, titled “LINKING PATIENT-SPECIFIC MEDICAL DEVICES WITH PATIENT-SPECIFIC DATA, AND ASSOCIATED SYSTEMS, DEVICES, AND METHODS;”
U.S. application Ser. No. 17/085,564, filed Oct. 30, 2020, titled “SYSTEMS AND METHODS FOR DESIGNING ORTHOPEDIC IMPLANTS BASED ON TISSUE CHARACTERISTICS;”
U.S. application Ser. No. 17/100,396, filed Nov. 20, 2020, titled “PATIENT-SPECIFIC VERTEBRAL IMPLANTS WITH POSITIONING FEATURES;”
U.S. application Ser. No. 17/342,439, filed Jun. 8, 2021, titled “PATIENT-SPECIFIC MEDICAL PROCEDURES AND DEVICES, AND ASSOCIATED SYSTEMS AND METHODS;”
U.S. application Ser. No. 17/463,054, filed Aug. 31, 2021, titled “BLOCKCHAIN MANAGED MEDICAL IMPLANTS;”
U.S. application Ser. No. 17/518,524, filed Nov. 3, 2021, titled “PATIENT-SPECIFIC ARTHROPLASTY DEVICES AND ASSOCIATED SYSTEMS AND METHODS;”
U.S. application Ser. No. 17/531,417, filed Nov. 19, 2021, titled “PATIENT-SPECIFIC JIG FOR PERSONALIZED SURGERY;”
U.S. application Ser. No. 17/678,874, filed Feb. 23, 2022, titled “NON-FUNGIBLE TOKEN SYSTEMS AND METHODS FOR STORING AND ACCESSING HEALTHCARE DATA;”
U.S. application Ser. No. 17/835,777, filed Jun. 8, 2022, titled “PATIENT-SPECIFIC EXPANDABLE INTERVERTEBRAL IMPLANTS;”
U.S. application Ser. No. 17/842,242, filed Jun. 16, 2022, titled “PATIENT-SPECIFIC ANTERIOR PLATE IMPLANTS;”
U.S. application Ser. No. 17/851,487, filed Jun. 28, 2022, titled “PATIENT-SPECIFIC ADJUSTMENT OF SPINAL IMPLANTS, AND ASSOCIATED SYSTEMS AND METHODS;”
U.S. application Ser. No. 17/856,625, filed Jul. 1, 2022, titled “SPINAL IMPLANTS FOR MESH NETWORKS;”
U.S. application Ser. No. 17/867,621, filed Jul. 18, 2022, titled “PATIENT-SPECIFIC SACROILIAC IMPLANT, AND ASSOCIATED SYSTEMS AND METHODS;”
U.S. application Ser. No. 17/868,729, filed Jul. 19, 2022, titled “SYSTEMS FOR PREDICTING INTRAOPERATIVE PATIENT MOBILITY AND IDENTIFYING MOBILITY-RELATED SURGICAL STEPS;”
U.S. application Ser. No. 17/951,085, filed Sep. 22, 2022, titled “SYSTEMS FOR MANUFACTURING AND PRE-OPERATIVE INSPECTING OF PATIENT-SPECIFIC IMPLANTS;”
U.S. application Ser. No. 17/978,673, filed Nov. 1, 2022, titled “SPINAL IMPLANTS AND SURGICAL PROCEDURES WITH REDUCED SUBSIDENCE, AND ASSOCIATED SYSTEMS AND METHODS;”
U.S. application Ser. No. 17/978,746, filed Nov. 1, 2022, titled “PATIENT-SPECIFIC SPINAL INSTRUMENTS FOR IMPLANTING IMPLANTS AND DECOMPRESSION PROCEDURES;”
U.S. application Ser. No. 18/102,444, filed Jan. 27, 2023, titled “TECHNIQUES TO MAP THREE-DIMENSIONAL HUMAN ANATOMY DATA TO TWO-DIMENSIONAL HUMAN ANATOMY DATA;”
U.S. application Ser. No. 18/113,573, filed Feb. 24, 2023, titled “PATIENT-SPECIFIC IMPLANT DESIGN AND MANUFACTURING SYSTEM WITH A DIGITAL FILING CABINET;”
U.S. application Ser. No. 18/120,979, filed Mar. 13, 2023, titled “MULTI-STAGE PATIENT-SPECIFIC SURGICAL PLANS AND SYSTEMS AND METHODS FOR CREATING AND IMPLEMENTING THE SAME;”
U.S. application Ser. No. 18/455,881, filed Aug. 25, 2023, titled “SYSTEMS AND METHODS FOR GENERATING MULTIPLE PATIENT-SPECIFIC SURGICAL PLANS AND MANUFACTURING PATIENT-SPECIFIC IMPLANTS;”
U.S. application Ser. No. 18/384,762, filed Oct. 28, 2023, titled “SYSTEMS AND METHODS FOR SELECTING, REVIEWING, MODIFYING, AND/OR APPROVING SURGICAL PLANS;”
U.S. application Ser. No. 18/537,600, filed Dec. 12, 2023, titled “PATIENT-SPECIFIC IMPLANT DESIGN AND MANUFACTURING SYSTEM WITH A REGULATORY AND REIMBURSEMENT MANAGER;”
International Application No. PCT/US24/10202, filed Jan. 3, 2024, titled “PATIENT-SPECIFIC SPINAL FUSION DEVICES AND ASSOCIATED SYSTEMS AND METHODS;”
U.S. application Ser. No. 18/408,409, filed Jan. 9, 2024, titled “SYSTEM FOR EDGE CASE PATHOLOGY IDENTIFICATION AND IMPLANT MANUFACTURING;”
U.S. application Ser. No. 18/408,452, filed Jan. 9, 2024, titled “SYSTEM FOR MODELING PATIENT SPINAL CHANGES;”
U.S. application Ser. No. 18/415,577, filed Jan. 17, 2024, titled “PATIENT-SPECIFIC IMPLANT DESIGN AND MANUFACTURING SYSTEM WITH A SURGICAL IMPLANT POSITIONING MANAGER;” and
U.S. application Ser. No. 18/892,151, filed Sep. 20, 2024, titled “ROTATABLE AND SURGICAL APPROACH-SPECIFIC INTERVERTEBRAL IMPLANTS FOR FUSION TECHNIQUES.”

All of the above-identified patents and applications are incorporated by reference in their entireties. In addition, the embodiments, features, systems, devices, materials, methods and techniques described herein may, in certain embodiments, be applied to or used in connection with any one or more of the embodiments, features, systems, devices, or other matter.

The ranges disclosed herein also encompass any and all overlap, sub-ranges, and combinations thereof. Language such as “up to,” “at least,” “greater than,” “less than,” “between,” or the like includes the number recited. Numbers preceded by a term such as “approximately,” “about,” and “substantially” as used herein include the recited numbers (e.g., about 10%=10%), and also represent an amount close to the stated amount that still performs a desired function or achieves a desired result. For example, the terms “approximately,” “about,” and “substantially” may refer to an amount that is within less than 10% of, within less than 5% of, within less than 1% of, within less than 0.1% of, and within less than 0.01% of the stated amount.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting.

I/We claim:

1. A medical treatment system for treating a patient, the system comprising:
a patient-specific interbody implant having at least one implant surface designed to mate with a first corresponding surface of a first vertebral body of the patient when the patient-specific interbody implant is implanted at a first predetermined target location between the first vertebral body and an adjacent second vertebral body of the patient;
an inserter instrument having an elongated shaft extending between a proximal complex and a distal end region, wherein the distal end region is sized and shaped to be coupled to the patient-specific interbody implant; and
a patient-specific positioning guide configured to be coupled to the elongated shaft proximate the patient-specific interbody implant, wherein the patient-specific positioning guide includes one or more contoured surfaces designed to mate with a second corresponding surface of the first vertebral body and/or the second vertebral body when the patient-specific positioning guide is implanted at a second predetermined target location,
wherein, when the patient-specific positioning guide and the patient-specific interbody implant are coupled to the elongated shaft, placing the patient-specific positioning guide at the second predetermined target location automatically places the patient-specific interbody implant at the first predetermined target location.

2. The system of claim 1 wherein the patient-specific positioning guide includes:
a first contoured surface designed to mate with the second corresponding surface, wherein the second corresponding surface is on the first vertebral body; and
a second contoured surface designed to mate with a third corresponding surface on the second vertebral body such that the patient-specific positioning guide is configured to contact both the first vertebral body and the second vertebral body.

3. The system of claim 1 wherein the second corresponding surface is at an anterior, lateral, or posterior margin of the first and/or second vertebral body.

4. The system of claim 1 wherein the patient-specific positioning guide includes:
a body having the one or more contoured surfaces;
a depth stop extending from the body; and
a lumen extending through the body and the depth stop.

5. The system of claim 4 wherein a length of the depth stop is based at least in part on a distance between the second predetermined target position and the first predetermined target position.

6. The system of claim 4 wherein the depth stop has a height less than a distance between the first vertebral body and the second vertebral body.

7. The system of claim 1 wherein the patient-specific positioning guide is configured to control one or more dimensions associated with the patient-specific interbody implant and the first predetermined target location, the one or more dimensions including (a) a distance between an entry point and the patient-specific interbody implant, (b) a distance between an entry point and a midline of the first vertebral body, (c) an axial angle of the patient-specific interbody implant, (d) a distance between the second vertebral body and a midline of the patient-specific interbody implant, and/or (e) a lateral angel of the patient-specific interbody implant.

8. The system of claim 1, further comprising a virtual model showing the first vertebral body, the second vertebral body, a virtual patient-specific interbody implanted at the first predetermined target location, and a virtual positioning guide implanted at the second predetermined target location.

9. The system of claim 8 wherein the patient-specific positioning guide includes one or more radiographic markers that can be imaged and compared to the virtual model to determine whether the patient-specific positioning guide is at the second predetermined target location and/or that the patient-specific interbody implant is at the first predetermined target location.

10. The system of claim 1, further comprising a camera configured to be coupled to the distal end region of the elongated shaft to provide real-time image and/or video feedback of a position of the patient-specific positioning guide and/or the patient-specific interbody implant.

11. The system of claim 1 wherein the patient-specific interbody implant is composed of a metal or metal alloy, and wherein the patient-specific positioning guide is composed of plastic.

12. The system of claim 1 wherein the patient-specific positioning guide is configured to remain coupled to the inserter instrument after the patient-specific interbody implant is released from the inserter instrument at the first predetermined target location.

13. A patient-specific positioning guide for use with implanting a patient-specific interbody implant into a patient, the patient-specific positioning guide comprising:
a first contoured surface designed to mate with a first surface of a first vertebral body of the patient, the first surface being at a lateral, anterior, or posterior margin of the first vertebral body;
a second contoured surface designed to mate with a second surface of a second vertebral body of the patient, the second surface being at a lateral, anterior, or posterior margin of the second vertebral body and the second vertebral body being immediately adjacent to the first vertebral body; and
a connection mechanism for connecting the patient-specific positioning guide to an inserter instrument at a predetermined orientation,
wherein the patient-specific positioning guide is designed such that when (a) the patient-specific positioning guide is coupled to the inserter instrument at the predetermined orientation, and (b) the first contoured surface mates with the first surface and the second contoured surface mates with the second surface, the patient-specific interbody implant is directed to a target implant location.

14. The patient-specific positioning guide of claim 13 wherein the patient-specific positioning guide includes a body and a depth stop extending from the body, wherein the body has the first contoured surface and the second contoured surface.

15. The patient-specific positioning guide of claim 14 wherein the body and the depth stop include a lumen extending therethrough, wherein the lumen is sized and shaped to rotatably receive an inner shaft of the inserter instrument.

16. The patient-specific positioning guide of claim 13 wherein the patient-specific positioning guide is a unitary component.

17. The patient-specific positioning guide of claim 13 wherein the patient-specific positioning guide is composed of plastic.

18. The patient-specific positioning guide of claim 13 wherein the connection mechanism is configured to remain coupled to the inserter instrument following deployment of the patient-specific interbody implant.

19. The patient-specific positioning guide of claim 13 wherein the patient-specific positioning guide is designed to control (a) an insertion depth of the patient-specific interbody implant, and/or (b) an insertion angle of the patient-specific interbody implant, such that, when implanted, the patient-specific interbody implant is directed to the target implant location.

20. The patient-specific positioning guide of claim 13 wherein the patient-specific positioning guide is designed to control an axial angle and/or a lateral angle of the patient-specific interbody implant such that, when implanted, the patient-specific interbody implant is directed to the target implant location.

21. A computer-implemented method for providing patient-specific treatment for a patient, the method comprising:
generating a virtual model of the patient's spine, wherein the virtual model includes one or more virtual surgical corrections to the patient's spine;
based at least in part on the virtual model showing the one or more virtual surgical corrections, designing a patient-specific interbody implant for achieving anatomical corrections that correspond to the one or more virtual surgical corrections, wherein designing the patient-specific interbody implant includes identifying a first target location for the patient-specific implant to be implanted between a first vertebral body and a second vertebral body;
based on the patient-specific implant and the first target location, designing a patient-specific positioning guide configured to be coupled to an inserter instrument for use with implanting the patient-specific interbody implant, wherein designing the patient-specific positioning guide includes:
designing one or more contoured surfaces of the patient-specific positioning guide to mate with a surface of the first vertebral body and/or the second vertebral body when the patient-specific positioning guide is positioned at a second target location, and
designing a size and/or shape of the patient-specific positioning guide such that, when the patient-specific positioning guide and the patient-specific interbody implant are coupled to the inserter instrument, placing the patient-specific positioning guide at the second target location automatically places the patient-specific interbody implant at the first target location.

22. The method of claim 21 further comprising generating manufacturing instructions for the patient-specific interbody implant and the patient-specific positioning guide.

23. The method of claim 21 further comprising manufacturing the patient-specific interbody implant and the patient-specific positioning guide.

24. The method of claim 21 wherein generating the virtual model includes manipulating one or more spatial relationships between vertebrae in the virtual model to provide the one or more virtual surgical corrections to the patient's spine.

25. The method of claim 21 wherein designing the patient-specific positioning guide includes designing a size and/or shape of the positioning guide to control an insertion depth of the patient-specific interbody implant.

26. The method of claim 21 wherein designing the patient-specific positioning guide includes designing a size and/or shape of the positioning guide to control an insertion angle of the patient-specific interbody implant.

27. The method of claim 21 wherein the patient-specific positioning guide is designed to remain coupled to the inserter instrument following deployment of the patient-specific interbody implant.
